(19) United States
(12) Patent Application Publication
(10) Pub. No.: US 2018/0282756 A1
(43) Pub. Date: Oct. 4, 2018

(54) PRE-MRNA PROCESSING FACTOR 19 (PRP19) NUCLEIC ACID MOLECULES TO CONTROL INSECT PESTS

(71) Applicants: Dow AgroSciences LLC, Indianapolis, IN (US); Fraunhofer-Gesellschaft zur Förderung der angewandten Forschung e.V., München (DE)

(72) Inventors: Kenneth E. Narva, Zionsville, IN (US); Chaoxian Geng, Zionsville, IN (US); Meghan Frey, Greenwood, IN (US); Premchand Gandra, Zionsville, IN (US); Andreas Vilcinskas, Giessen (DE); Catherine D. Young, Indianapolis, IN (US); Abhilash Balachandran, Carmel, IN (US); Eileen Knorr, Gieben (DE); Rainer Fischer, Aachen (DE)

(21) Appl. No.: 15/927,672

(22) Filed: Mar. 21, 2018

Related U.S. Application Data
(60) Provisional application No. 62/474,509, filed on Mar. 21, 2017.

(51) Int. Cl. C12N 15/82; C12N 15/113; C12N 15/89

(52) U.S. Cl. CPC C12N 15/8286; C12N 15/113; C12N 2310/14; C12N 15/8285; C12N 15/8201; C12N 15/89

(57) ABSTRACT

This disclosure concerns nucleic acid molecules and methods of use thereof for control of insect pests through RNA interference-mediated inhibition of target coding and transcribed non-coding sequences in insect pests, including pollen beetle. The disclosure also concerns methods for making transgenic plants that express nucleic acid molecules useful for the control of insect pests, and the plant cells and plants obtained thereby.

### PRIORITY CLAIM

This application claims the benefit of U.S. Provisional Patent Application No. 62/474,509, filed on Mar. 21, 2017, the entirety of which is incorporated herein.

### TECHNICAL FIELD OF THE DISCLOSURE

The present invention relates generally to genetic control of plant damage caused by insect pests (e.g., pollen beetle). In particular embodiments, the present invention relates to identification of target coding and non-coding polynucleotides, and the use of recombinant DNA technologies for post-transcriptionally repressing or inhibiting expression of target coding and non-coding polynucleotides in the cells of an insect pest to provide a plant protective effect.

### BACKGROUND

European pollen beetles (PB) are serious pests in oilseed rape, both the larvae and adults feed on flowers and pollen. Pollen beetle damage to the crop can cause 20-40% yield loss. The primary pest species is Meligethes aeneus. Currently, pollen beetle control in oilseed rape relies mainly on pyrethroids which are expected to be phased out soon because of their environmental and regulatory profile. Moreover, pollen beetle resistance to existing chemical insecticides has been reported. Therefore, urgently needed are environmentally friendly pollen beetle control solutions with novel modes of action.

In nature, pollen beetles overwinter as adults in the soil or under leaf litter. In spring the adults emerge from hibernation and start feeding on flowers of weeds, and migrate onto flowering oilseed rape plants. The eggs are laid in oilseed rape flower buds. The larvae feed and develop in the buds and on the flowers. Late stage larvae find a pupation site in the soil. The second generation of adults emerge in July and August and feed on various flowering plants before finding sites for overwintering.

RNA interference (RNAi) is a process utilizing endogenous cellular pathways, whereby an interfering RNA (iRNA) molecule (e.g., a dsRNA molecule) that is specific for all, or any portion of adequate size, of a target gene results in the degradation of the mRNA encoded thereby. In recent years, RNAi has been used to perform gene “knockdown” in a number of species and experimental systems; for example, Caenorhabditis elegans, plants, insect embryos, and cells in tissue culture. See, e.g., Fire et al. (1998) Nature 391:806-11; Martinez et al. (2002) Cell 110:563-74; McManus and Sharp (2002) Nature Rev. Genetics 3:737-47.

RNAi accomplishes degradation of mRNA through an endogenous pathway including the DICER protein complex. DICER cleaves long dsRNA molecules into short fragments of approximately 20 nucleotides, termed small interfering RNA (siRNA). The siRNA is unwound into two single-stranded RNAs: the passenger strand and the guide strand. The passenger strand is degraded, and the guide strand is incorporated into the RNA-induced silencing complex (RISC).

The authors of U.S. Pat. No. 7,612,194 and U.S. Patent Publication No. 2007/0050860 demonstrated the potential for in planta RNAi as a possible pest management tool within the context of providing plant protection against western corn rootworm (D. v. virgifera LeConte), while simultaneously demonstrating that effective RNAi targets cannot be accurately identified a priori, even from a relatively small set of candidate genes. Baum et al. (2007) Nat. Biotechnol. 25(11):1322-6. Using a high-throughput in vivo dietary RNAi system to screen potential target genes for developing transgenic RNAi maize, these researchers found that, of an initial gene pool of 290 targets, only 14 exhibited larval control potential.

### SUMMARY OF THE DISCLOSURE

Disclosed herein are nucleic acid molecules (e.g., target genes, DNAs, dsRNAs, siRNAs, miRNAs, shRNAs, and hpRNAs), and methods of use thereof, for the control of insect pests, including, for example, Meligethes aeneus Fabricius (pollen beetle, “PB”). In particular examples, exemplary nucleic acid molecules are disclosed that may be homologous to at least a portion of one or more native nucleic acids in PB.

In these and further examples, the native nucleic acid sequence may be a target gene, the product of which may be, for example and without limitation: involved in a metabolic process; or involved in larval development. In some examples, post-transcriptional inhibition of the expression of a target gene by a nucleic acid molecule comprising a polynucleotide homologous thereto may be lethal to PB or result in reduced growth and/or development of PB. In specific examples, pre-myna processing factor 19 (referred to herein as prp19) or a prp19 homolog may be selected as a target gene for post-transcriptional silencing. In particular examples, a target gene useful for post-transcriptional inhibition is PBprp19; SEQ ID NO:1 (i.e., the PBprp19 polynucleotide characterized as comprising SEQ ID NOs:2-3). An isolated nucleic acid molecule comprising the polynucleotide of SEQ ID NO:1; the PB prp19 polynucleotide comprising SEQ ID NOs:2-3; fragments of PB prp19 (e.g., SEQ ID NOs:2-4); and/or the complement or reverse complement of any of the foregoing is therefore disclosed herein.

Also disclosed are nucleic acid molecules comprising a polynucleotide that encodes a polypeptide that is at least about 85% identical to an amino acid sequence within a target gene product (for example, the product of PBprp19). For example, a nucleic acid molecule may comprise a polynucleotide encoding a polypeptide that is at least 85% identical to PB PRP19; SEQ ID NO:5 (i.e., the PRP19 polypeptide characterized as comprising SEQ ID NOs:6-7); and/or an amino acid sequence within a product of a prp19 gene (e.g., SEQ ID NOs:6-7). Further disclosed are nucleic acid molecules comprising a polynucleotide that is the complement or reverse complement of a polynucleotide that encodes a polypeptide at least 85% identical to an amino acid sequence within a target gene product.

Also disclosed are cDNA polynucleotides that may be used for the production of iRNA (e.g., dsRNA, siRNA, shRNA, miRNA, and hpRNA) molecules that are complementary to all or part of an insect pest target gene, for example, aprp19 gene. In particular embodiments, dsRNAs, siRNAs, shRNAs, miRNAs, and/or hpRNAs may be produced in vitro, or in vivo by a genetically-modified organism, such as a plant or bacterium. In particular examples, cDNA molecules are disclosed that may be used to produce iRNA molecules that are complementary or reverse complementary to all or part of prp19 (e.g., SEQ ID NO:1, the PBprp19 polynucleotide characterized as comprising SEQ ID NOs:2-3), or a fragment thereof.

Further disclosed are means for inhibiting expression of a prp19 gene in a Meligethes pest, and means for providing prp19-mediated Meligethes pest protection to a plant. A means for inhibiting expression of a prp19 gene in a Meligethes pest is a double-stranded RNA molecule, wherein one strand of the molecule consists of the polyribonucleotide of SEQ ID NO:15; and the complements thereof. Functional equivalents of means for inhibiting expression of aprp19 gene in a Meligethes pest include double-stranded RNA molecules comprising a polyribonucleotide that is substantially homologous to all or part of the Meligethes aeneus Fabricius prp19 gene comprising SEQ ID NOs:2-3. A means for providing prp19-mediated Meligethes pest protection to a plant is a DNA molecule comprising a polynucleotide encoding a means for inhibiting expression of a prp19 gene in a Meligethes pest operably linked to a promoter functional in a plant cell (e.g., a canola cell).

Additionally, disclosed are methods for controlling a population of an insect pest (e.g., pollen beetle), comprising providing to an insect pest an iRNA (e.g., dsRNA, siRNA, shRNA, miRNA, and hpRNA) molecule that functions upon being taken up by the pest to inhibit a biological function within the pest. In some embodiments, the iRNA molecule that functions upon being taken up by the pest to inhibit a biological function within the pest comprises all or part of a polyribonucleotide selected from the group consisting of: SEQ ID NO:12; the complement or reverse complement of SEQ ID NO:12; SEQ ID NO:13; the complement or reverse complement of SEQ ID NO:13; SEQ ID NO:14; the complement or reverse complement of SEQ ID NO:14; the native polyribonucleotide from PB that comprises SEQ ID NOs:13-14; the complement or reverse complement of the native polyribonucleotide from PB that comprises SEQ ID NOs:13-14; SEQ ID NO:15; the complement or reverse complement of SEQ ID NO:15; a polyribonucleotide that hybridizes to the transcript of a native coding polynucleotide of a Meligethes organism (e.g., PB) comprising all or part of any of SEQ ID NOs:2-4; and the complement or reverse complement of a polyribonucleotide that hybridizes to the transcript of a native coding polynucleotide of a Meligethes organism comp comprising all or part of any of SEQ ID NOs:2-4.

In particular embodiments, an iRNA that functions upon being taken up by an insect pest to inhibit a biological function within the pest is transcribed from a DNA comprising all or part of a polynucleotide selected from the group consisting of: SEQ ID NO:1; the complement or reverse complement of SEQ ID NO:1; the native coding polynucleotide from PB that comprises SEQ ID NOs:2-3; the complement of the native coding polynucleotide from PB that comprises SEQ ID NOs:2-3; SEQ ID NO:4; the complement or reverse complement of SEQ ID NO:4; a native coding polynucleotide of a Meligethes organism comprising all or part of any of SEQ ID NOs:2-4; and the complement or reverse complement of a native coding polynucleotide of a Meligethes organism comprising all or part of any of SEQ ID NOs:2-4.

Also disclosed herein are methods wherein dsRNAs, siRNAs, shRNAs, miRNAs, and/or hpRNAs may be provided to an insect pest in a diet-based assay, or in genetically-modified plant cells expressing the dsRNAs, siRNAs, shRNAs, miRNAs, and/or hpRNAs. In these and further examples, the dsRNAs, siRNAs, shRNAs, miRNAs, and/or hpRNAs may be ingested by the pest. Ingestion of dsRNAs, siRNA, shRNAs, miRNAs, and/or hpRNAs of the invention may then result in RNAi in the pest, which in turn may result in silencing of a gene essential for viability of the pest and leading ultimately to mortality. In particular examples, an insect pest controlled by use of nucleic acid molecules of the invention may be pollen beetle (Meligethes aeneus).

The foregoing and other features will become more apparent from the following Detailed Description of several embodiments, which proceeds with reference to the accompanying FIGS. 1-2.

### BRIEF DESCRIPTION OF THE FIGURES

FIG. 1 includes a depiction of a strategy used to provide dsRNA from a single transcription template with a single pair of primers.

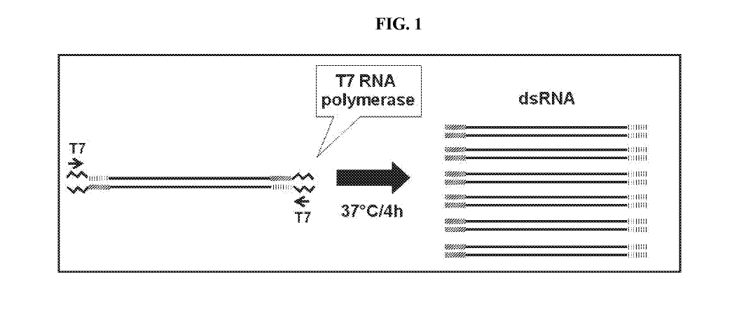

FIG. 2 includes a depiction of a strategy used to provide dsRNA from two transcription templates.

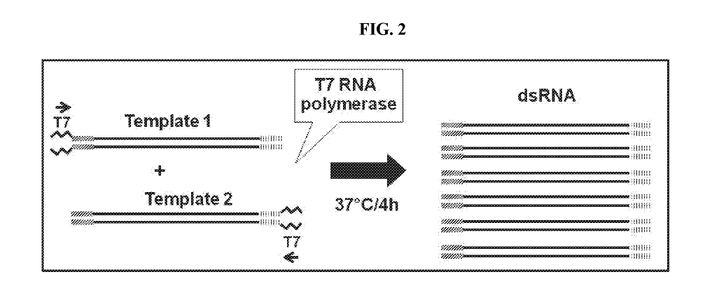

### SEQUENCE LISTING

The nucleic acid sequences listed in the accompanying sequence listing are shown using standard letter abbreviations for nucleotide bases, as defined in 37 C.F.R. § 1.822. The nucleotide and amino acid sequences listed define molecules (i.e., polynucleotides and polyribonucleotides, and polypeptides, respectively) having the nucleotide and amino acid monomers arranged in the manner described. The nucleotide and amino acid sequences listed also each define a genus of polynucleotides/polyribonucleotides or polypeptides that comprise the nucleotide and amino acid monomers arranged in the manner described. In view of the redundancy of the genetic code, it is understood by those in the art that a nucleotide sequence including a coding sequence also describes the genus of polynucleotides encoding the same polypeptide as a polynucleotide consisting of the reference sequence. It is further understood that an amino acid sequence describes the genus of polynucleotide ORFs encoding that polypeptide.

Only one strand of each nucleotide sequence is shown, but the complementary strand is included by any reference to the displayed strand. As the complement and reverse complement of a primary nucleic acid sequence are necessarily disclosed by the primary sequence, the complementary sequence and reverse complementary sequence of a nucleotide sequence are included by any reference to the nucleotide sequence, unless it is explicitly stated to be otherwise (or it is clear to be otherwise from the context in which the sequence appears). Furthermore, as it is understood in the art that the ribonucleotide sequence of an RNA strand is determined by the sequence of the DNA from which it was transcribed (but for the substitution of uracil (U) nucleobases for thymine (T)), an RNA sequence is included by any reference to the DNA sequence encoding it. In the accompanying sequence listing:

SEQ ID NO:1 shows an exemplary pollen beetle (Meligethes aeneus) prp19 DNA, referred to herein in some places as PB prp19:

ATGGCGTTGTGTTGTGCCATTTCGAACGAAGTTCCGGAGCATCCAGTGGT
TTCTCCTTCATCCGGAGTAATATTCGAGAGAAGAATAATCGAAAAATACA
TACAGGAGAATGGAGTGGACCCTATAAGTGGAAAAGAAGTCGCTATAGAT
GAATTAATAGAAATCAAAACGCCTCCAATTGTAAAACCAAAGCCACCAAG
TGCTACTAGCATCCCAGCTACATTAAAACTACTGCAGGATGAATGGGATG
CGGTTATGCTGTACAGTTTTACACAACGGCAGCAATTACAAACAGCGCGA
CAGGAATTATCGCACGCTTTATATCAACACGATGCTGCCTGCAGGGTGAT
TGCAAGGTTAAACAAGGAAGTAACTGCTGCAAGAGAGGCTCTGGCAACAT
TGAAACCACAAGCTGGTATTACTACAGTCCCTCAACCTGCTGTTGCTGCT
GAAGCTGGCGGTGTTGCCAATCAACCCACTGAACAAGCTGGCATGAGTGT
GGAGGTTATACAAAAATTGCAAGATAAGGCAGCCGTTCTGACACAGGAGC
GTAAAAAGAGGGGTCGCACAGTTCCAGAAGAGTTGACTACTCAGGAACAG
TTGAGGAGTTTCAGGACATTGGCATCCCTTATTGGTCTTCATTCCGCCAG
TATTCCTGGTATTTTAGCTTTAGATGTGCACAGTTCAGATACCAGTAAAG
TATTAACAGGAGGAAACGACAAAAATGCTACTGTATTCAACAAAGATACC
GAACAGGTCGTCACAATTTTAAAAGGACACACTAAAAAAGTGACAAAGGT
CATTTACCATCCCGAAGAAGATATTGTTATTACAGCTTCTCCCGATTCGA
CAATCAGAGTTTGGAACGTGCCGACCTCGCAAACCACTCTCCTCNNNNNN
NNNNNNNNNNNNNNNNNNNNNNNNNNNTCCCTGCACCCCACGGGAGACTA
CGTTCTGTCCACTTCCGAAGACCAACATTGGGCGTTTTCCGACATCCGTA
CCGGCAGACTTCTGACCAAAGTTTCCGATCAATCGAACGTTCCGCTGACC
ACCGCGCAGCTGCATCCCGACGGTCTCATCTTCGGCACCGGAACCGGGGA
TTCCCAGGTCAAAATTTGGGATTTGAAGGAGCAGAGCAACGTCGCCAACT
TCACAGGACATTCCGGAGCAATCACCACGATTTCCTTCTCGGAAAACGGG
TATTACTTGGCCACTGCCGCCGACGACGCGTGCGTAAAACTATGGGATTT
GCGTAAATTAAGAAACTTCAAAACTCTACAACTCGACGAAGGTTACCAAA
TCAAAGATTTGTGTTTCGATCAAAGCGGAACCTATTTGGCCGTTGCCGGA
ACTGACGTCAGGGTTTANNNNNNNNNNNNNNNNNNNNNNNNNNNNNNNNN

SEQ ID NO:2 shows a characteristic fragment of an exemplary pollen beetle prp19 DNA:

ATGGCGTTGTGTTGTGCCATTTCGAACGAAGTTCCGGAGCATCCAGTGGT
TTCTCCTTCATCCGGAGTAATATTCGAGAGAAGAATAATCGAAAAATACA
TACAGGAGAATGGAGTGGACCCTATAAGTGGAAAAGAAGTCGCTATAGAT
GAATTAATAGAAATCAAAACGCCTCCAATTGTAAAACCAAAGCCACCAAG
TGCTACTAGCATCCCAGCTACATTAAAACTACTGCAGGATGAATGGGATG
CGGTTATGCTGTACAGTTTTACACAACGGCAGCAATTACAAACAGCGCGA
CAGGAATTATCGCACGCTTTATATCAACACGATGCTGCCTGCAGGGTGAT
TGCAAGGTTAAACAAGGAAGTAACTGCTGCAAGAGAGGCTCTGGCAACAT
TGAAACCACAAGCTGGTATTACTACAGTCCCTCAACCTGCTGTTGCTGCT
GAAGCTGGCGGTGTTGCCAATCAACCCACTGAACAAGCTGGCATGAGTGT
GGAGGTTATACAAAAATTGCAAGATAAGGCAGCCGTTCTGACACAGGAGC
GTAAAAAGAGGGGTCGCACAGTTCCAGAAGAGTTGACTACTCAGGAACAG
TTGAGGAGTTTCAGGACATTGGCATCCCTTATTGGTCTTCATTCCGCCAG
TATTCCTGGTATTTTAGCTTTAGATGTGCACAGTTCAGATACCAGTAAAG
TATTAACAGGAGGAAACGACAAAAATGCTACTGTATTCAACAAAGATACC
GAACAGGTCGTCACAATTTTAAAAGGACACACTAAAAAAGTGACAAAGGT
CATTTACCATCCCGAAGAAGATATTGTTATTACAGCTTCTCCCGATTCGA
CAATCAGAGTTTGGAACGTGCCGACCTCGCAAACCACTCTCCTC

SEQ ID NO:3 shows a further characteristic fragment of an exemplary pollen beetle prp19 DNA:

TCCCTGCACCCCACGGGAGACTACGTTCTGTCCACTTCCGAAGACCAACA
TTGGGCGTTTTCCGACATCCGTACCGGCAGACTTCTGACCAAAGTTTCCG
ATCAATCGAACGTTCCGCTGACCACCGCGCAGCTGCATCCCGACGGTCTC
ATCTTCGGCACCGGAACCGGGGATTCCCAGGTCAAAATTTGGGATTTGAA
GGAGCAGAGCAACGTCGCCAACTTCACAGGACATTCCGGAGCAATCACCA
CGATTTCCTTCTCGGAAAACGGGTATTACTTGGCCACTGCCGCCGACGAC
GCGTGCGTAAAACTATGGGATTTGCGTAAATTAAGAAACTTCAAAACTCT
ACAACTCGACGAAGGTTACCAAATCAAAGATTTGTGTTTCGATCAAAGCG
GAACCTATTTGGCCGTTGCCGGAACTGACGTCAGGGTTTA

SEQ ID NO:4 shows a further exemplary Meligethes prp19 DNA, referred to herein in some places as PB prp19 regi (region 1), which is used in some examples for the production of a dsRNA:

GGCGTTGTGTTGTGCCATTTCGAACGAAGTTCCGGAGCATCCAGTGGTTT
CTCCTTCATCCGGAGTAATATTCGAGAGAAGAATAATCGAAAAATACATA
CAGGAGAATGGAGTGGACCCTATAAGTGGAAAAGAAGTCGCTATAGATGA
ATTAATAGAAATCAAAACGCCTCCAATTGTAAAACCAAAGCCACCAAGTG
CTACTAGCATCCCAGCTACATTAAAACTACTGCAGGATGAATGGGATGCG
GTTATGCTGTACAGTTTTACACAACGGCAGCAATTACAAACAGCGCGACA
GGAATT

SEQ ID NO:5 shows the amino acid sequence of a Meligethes PRP19 polypeptide encoded by an exemplary PB prp19 DNA:

MALCCAISNEVPEHPVVSPSSGVIFERRIIEKYIQENGVDPISGKEVAID
ELIEIKTPPIVKPKPPSATSIPATLKLLQDEWDAVMLYSFTQRQQLQTAR
QELSHALYQHDAACRVIARLNKEVTAAREALATLKPQAGITTVPQPAVAA
EAGGVANQPTEQAGMSVEVIQKLQDKAAVLTQERKKRGRTVPEELTTQEQ
LRSFRTLASLIGLHSASIPGILALDVHSSDTSKVLTGGNDKNATVFNKDT
EQVVTILKGHTKKVTKVIYHPEEDIVITASPDSTIRVWNVPTSQTTLLXX
XXXXXXXXXSLHPTGDYVLSTSEDQHWAFSDIRTGRLLTKVSDQSNVPLT
TAQLHPDGLIFGTGTGDSQVKIWDLKEQSNVANFTGHSGAITTISFSENG
YYLATAADDACVKLWDLRKLRNFKTLQLDEGYQIKDLCFDQSGTYLAVAG
TDVRVXXXXXXXXXXXX

SEQ ID NO:6 shows a characteristic amino acid sequence of a Meligethes PRP19 polypeptide:

MALCCAISNEVPEHPVVSPSSGVIFERRIIEKYIQENGVDPISGKEVAID
ELIEIKTPPIVKPKPPSATSIPATLKLLQDEWDAVMLYSFTQRQQLQTAR
QELSHALYQHDAACRVIARLNKEVTAAREALATLKPQAGITTVPQPAVAA
EAGGVANQPTEQAGMSVEVIQKLQDKAAVLTQERKKRGRTVPEELTTQEQ
LRSFRTLASLIGLHSASIPGILALDVHSSDTSKVLTGGNDKNATVFNKDT
EQVVTILKGHTKKVTKVIYHPEEDIVITASPDSTIRVWNVPTSQTTLL

SEQ ID NO:7 shows a further characteristic amino acid sequence of a Meligethes PRP19 polypeptide:

SLHPTGDYVLSTSEDQHWAFSDIRTGRLLTKVSDQSNVPLTTAQLHPDGL
IFGTGTGDSQVKIWDLKEQSNVANFTGHSGAITTISFSENGYYLATAADD
ACVKLWDLRKLRNFKTLQLDEGYQIKDLCFDQSGTYLAVAGTDVRV

SEQ ID NO:8 shows a nucleotide sequence of T7 phage promoter.

SEQ ID NOs:9-10 show primers used for PCR amplification of prp19 sequences comprising PB prp19 regi, used in some examples for dsRNA production.

SEQ ID NO:11 shows an exemplary DNA encoding a PB prp19 regi hairpin-forming RNA, containing a sense nucleotide sequence, a loop sequence comprising an intron (underlined), and an antisense nucleotide sequence (bold font):

GGCGTTGTGTTGTGCCATTTCGAACGAAGTTCCGGAGCATCCAGTGGTT
TCTCCTTCATCCGGAGTAATATTCGAGAGAAGAATAATCGAAAAATACA
TACAGGAGAATGGAGTGGACCCTATAAGTGGAAAAGAAGTCGCTATAGA
TGAATTAATAGAAATCAAAACGCCTCCAATTGTAAAACCAAAGCCACCA
AGTGCTACTAGCATCCCAGCTACATTAAAACTACTGCAGGATGAATGGG
ATGCGGTTATGCTGTACAGTTTTACACAACGGCAGCAATTACAAACAGC
GCGACAGGAATTGACTAGTACCGGTTGGGAAAGGTATGTTTCTGCTTCT
ACCTTTGATATATATATAATAATTATCACTAATTAGTAGTAATATAGTA
TTTCAAGTATTTTTTTCAAAATAAAAGAATGTAGTATATAGCTATTGCT
TTTCTGTAGTTTATAAGTGTGTATATTTTAATTTATAACTTTTCTAATA
TATGACCAAAACATGGTGATGTGCAGGTTGATCCGCGGTTAAATTCCTG
TCGCGCTGTTTGTAATTGCTGCCGTTGTGTAAAACTGTACAGCATAACC
GCATCCCATTCATCCTGCAGTAGTTTTAATGTAGCTGGGATGCTAGTAG
CACTTGGTGGCTTTGGTTTTACAATTGGAGGCGTTTTGATTTCTATTAA
TTCATCTATAGCGACTTCTTTTCCACTTATAGGGTCCACTCCATTCTCC
TGTATGTATTTTTCGATTATTCTTCTCTCGAATATTACTCCGGATGAAG
GAGAAACCACTGGATGCTCCGGAACTTCGTTCGAAATGGCACAACACAA
CGCC

SEQ ID NOs:12-31 show exemplary RNAs transcribed from exemplary prp19 polynucleotides and fragments thereof, and processed therefrom, for example, by DICER activity.

### DETAILED DESCRIPTION

### I. Overview of Several Embodiments

We developed RNA interference (RNAi) as a tool for insect pest management, using a likely target pest species for transgenic plants that express dsRNA; the European pollen beetle. Herein, we describe RNAi-mediated knockdown of pre-myna processing factor 19 (prp19) in the exemplary insect pest, Eurpoean pollen beetle, which is shown to have a lethal phenotype when, for example, iRNA molecules are delivered via ingested or injected prp19 dsRNA. In embodiments herein, the ability to deliver prp19 dsRNA by feeding to insects confers an RNAi effect that is very useful for insect pest management. By combining prp19-mediated RNAi with other useful RNAi targets, the potential to affect multiple target sequences (for example, to achieve synergistic control by inhibiting target sequences with multiple modes of action) increases the opportunities to develop sustainable approaches to insect pest management involving RNAi technologies.

Disclosed herein are methods and compositions for genetic control of insect (e.g., PB) pest infestations. Methods for identifying one or more gene(s) essential to the lifecycle of an insect pest for use as a target gene for RNAi-mediated control of an insect pest population are also provided. DNA plasmid vectors encoding an RNA molecule may be designed to suppress one or more target gene(s) essential for growth, survival, and/or development. In some embodiments, methods are provided for post-transcriptional repression of expression or inhibition of a target gene via nucleic acid molecules that are complementary to a coding or non-coding sequence of the target gene in an insect pest. In these and further embodiments, a pest may ingest one or more dsRNA, siRNA, shRNA, miRNA, and/or hpRNA molecules transcribed from all or a portion of a nucleic acid molecule that is complementary to a coding or non-coding sequence of a target gene, thereby providing a plant-protective effect. Thus, some embodiments involve sequence-specific inhibition of expression of target gene products, using dsRNA, siRNA, shRNA, miRNA and/or hpRNA that is complementary to coding and/or non-coding sequences of the target gene(s) to achieve at least partial control of an insect (e.g., coleopteran) pest. In some embodiments, a dsRNA molecule (e.g., SEQ ID NO:16) may be capable of forming miRNA or siRNA molecules of 21-23 ribonucleotides in length (e.g., SEQ ID NOs:17-31), for example, by processing of the dsRNA by the enzyme, DICER.

Disclosed are isolated and purified nucleic acid molecules characterized by a polynucleotide comprising at least one nucleotide sequence, for example, as set forth in SEQ ID NO:1 and SEQ ID NOs:2-3, fragments thereof, and the complements and reverse complements of the foregoing. In some embodiments, a stabilized dsRNA molecule may be expressed from these polynucleotides, fragments thereof, or a gene comprising one or more of these polynucleotides, for the post-transcriptional silencing or inhibition of a target gene. In certain embodiments, isolated and purified nucleic acid molecules comprise SEQ ID NO:1, all or part of the PB prp19 polynucleotide comprising SEQ ID NOs:2-3 (e.g., SEQ ID NO:4), and/or a complement or reverse complement thereof.

Some embodiments involve a recombinant host cell (e.g., a plant cell) having in its genome at least one recombinant DNA encoding at least one iRNA (e.g., dsRNA) molecule(s). In particular embodiments, an iRNA molecule may be provided when ingested by an insect pest to post-transcriptionally silence or inhibit the expression of a target gene in the pest. The recombinant DNA may comprise, for example, SEQ ID NO:1; all or part of the PB prp19 polynucleotide comprising SEQ ID NOs:2-3; fragments of the PB prp19 polynucleotide comprising SEQ ID NOs:2-3; SEQ ID NO:4; a polynucleotide consisting of a partial sequence of a gene comprising one of SEQ ID NOs:2-4; complements of the foregoing; and/or reverse complements of the foregoing.

Some embodiments involve a recombinant host cell having in its genome a recombinant DNA encoding at least one iRNA (e.g., dsRNA) molecule(s) comprising a ribonucleotide sequence selected from the group consisting of SEQ ID NO:12; all or part of the PB polyribonucleotide comprising SEQ ID NOs:13-14 (e.g., siRNAs and miRNAs consisting of at least one polyribonucleotide selected from the group characterized by SEQ ID NOs:17-31); and the complements and reverse complements of the foregoing. When ingested by an insect pest (e.g., PB), the iRNA molecule(s) may silence or inhibit the expression of a target prp19 DNA (e.g., a DNA comprising all or part of the PB prp19 polynucleotide comprising SEQ ID NOs:2-3, and SEQ ID NO:4) in the pest, and thereby result in cessation of growth, development, and/or feeding in the pest.

In some embodiments, a recombinant host cell having in its genome at least one recombinant DNA encoding at least one RNA molecule capable of forming a dsRNA molecule may be a transformed plant cell. Some embodiments involve transgenic plants comprising such a transformed plant cell. In addition to such transgenic plants, progeny plants of any transgenic plant generation, transgenic seeds, and transgenic plant products, are all provided, each of which comprises recombinant DNA(s). In particular embodiments, an RNA molecule capable of forming a dsRNA molecule may be expressed in a transgenic plant cell. Therefore, in these and other embodiments, a dsRNA molecule may be isolated from a transgenic plant cell. In particular embodiments, the transgenic plant is a plant selected from the group comprising plants of the family Brassica (e.g., Brassica napus).

Some embodiments involve a method for modulating the expression of a target gene in an insect pest cell. In these and other embodiments, a nucleic acid molecule may be provided, wherein the nucleic acid molecule comprises a polynucleotide encoding an RNA molecule capable of forming a dsRNA molecule. In particular embodiments, a polynucleotide encoding an RNA molecule capable of forming a dsRNA molecule may be operatively linked to a promoter, and may also be operatively linked to a transcription termination sequence. In particular embodiments, a method for modulating the expression of a target gene in an insect pest cell may comprise: (a) transforming a plant cell with a vector comprising a polynucleotide encoding an RNA molecule capable of forming a dsRNA molecule; (b) culturing the transformed plant cell under conditions sufficient to allow for development of a plant cell culture comprising a plurality of transformed plant cells; (c) selecting for a transformed plant cell that has integrated the polynucleotide into its genome; and (d) determining that the selected transformed plant cell comprises the RNA molecule capable of forming a dsRNA molecule encoded by the polynucleotide. A plant may be regenerated from a plant cell that has the polynucleotide integrated in its genome and comprises the dsRNA molecule encoded by the polynucleotide.

Thus, also disclosed is a transgenic plant comprising a polynucleotide encoding a dsRNA molecule integrated in its genome, wherein the transgenic plant comprises the dsRNA molecule encoded by the polynucleotide. In particular embodiments, expression of the dsRNA molecule in the plant is sufficient to modulate the expression of a target gene in a cell of an insect pest that contacts the transformed plant or plant cell (for example, by feeding on the transformed plant, a part of the plant (e.g., leaves), or plant cell), such that growth and/or survival of the pest is inhibited. Transgenic plants disclosed herein may display resistance and/or enhanced tolerance to insect pest infestations. Particular transgenic plants may display resistance and/or enhanced protection from Meligethes aeneus Fabricius.

Also disclosed herein are methods for delivery of control agents, such as an iRNA molecule, to an insect pest. Such control agents may cause, directly or indirectly, an impairment in the ability of an insect pest population to feed, grow or otherwise cause damage in a host. In some embodiments, a method is provided comprising delivery of a stabilized dsRNA molecule to an insect pest to suppress at least one target gene in the pest, thereby causing RNAi and reducing or eliminating plant damage in a pest host. In some embodiments, a method of inhibiting expression of a target gene in the insect pest may result in cessation of growth, survival, and/or development in the pest.

In some embodiments, compositions (e.g., a topical composition) are provided that comprise an iRNA (e.g., dsRNA) molecule for use with plants, insects, and/or the environment of a plant or insect to achieve the elimination or reduction of an insect pest infestation. In particular embodiments, the composition may be a nutritional composition or food source to be fed to the insect pest. Some embodiments comprise making the nutritional composition or food source available to the pest. Ingestion of a composition comprising iRNA molecules may result in the uptake of the molecules by one or more cells of the pest, which may in turn result in the inhibition of expression of at least one target gene in cell(s) of the pest. Ingestion of or damage to a plant or plant cell by an insect pest infestation may be limited or eliminated in or on any host tissue or environment in which the pest is present by providing one or more compositions comprising an iRNA molecule in the host of the pest.

The compositions and methods disclosed herein may be used together in combinations with other methods and compositions for controlling damage by insect pests. For example, an iRNA molecule as described herein for protecting plants from insect pests may be used in a method comprising the additional use of one or more chemical agents effective against an insect pest, biopesticides effective against such a pest, crop rotation, recombinant genetic techniques that exhibit features different from the features of RNAi-mediated methods and RNAi compositions (e.g., recombinant production of proteins in plants that are harmful to an insect pest (e.g., Bt toxins and PIP-1 polypeptides (See U.S. Patent Publication No. US 2014/0007292 A1)), and recombinant expression of other iRNA molecules).

### II. Abbreviations

dsRNA double-stranded ribonucleic acid

EST expressed sequence tag

NCBI National Center for Biotechnology Information

gDNA genomic DNA

iRNA inhibitory ribonucleic acid

ORF open reading frame

RNAi ribonucleic acid interference

miRNA micro ribonucleic acid

shRNA short hairpin ribonucleic acid

siRNA small inhibitory ribonucleic acid

hpRNA hairpin ribonucleic acid

UTR untranslated region

PB Pollen beetle (Meligethes aeneus Fabricius)

PCR Polymerase chain reaction

qPCR quantative polymerase chain reaction

RISC RNA-induced Silencing Complex

SEM standard error of the mean

### III. Terms

In the description and tables which follow, a number of terms are used. In order to provide a clear and consistent understanding of the specification and claims, including the scope to be given such terms, the following definitions are provided:

Coleopteran pest: As used herein, the term “coleopteran pest” refers to pest insects of the order Coleoptera, and specifically includes pest insects in the genus Meligethes, which feed upon agricultural crops and crop products, including canola. In particular examples, a coleopteran pest is Meligethes aeneus Fabricius.

Contact (with an organism): As used herein, the term “contact with” or “uptake by” an organism (e.g., an insect pest), with regard to a nucleic acid molecule, includes internalization of the nucleic acid molecule into the organism, including, for example and without limitation: ingestion of the molecule by the organism (e.g., by feeding); contacting the organism with a composition comprising the nucleic acid molecule; and soaking of the organism with a solution comprising the nucleic acid molecule.

Contig: As used herein the term “contig” refers to a DNA sequence that is reconstructed from a set of overlapping DNA segments derived from a single genetic source.

Expression: As used herein, “expression” of a coding polynucleotide (for example, a gene or a transgene) refers to the process by which the coded information of a nucleic acid transcriptional unit (including, e.g., gDNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, northern blot, RT-PCR, western blot, or in vitro, in situ, or in vivo protein activity assay(s).

Genetic material: As used herein, the term “genetic material” includes all genes, and nucleic acid molecules, such as DNA and RNA.

Inhibition: As used herein, the term “inhibition,” when used to describe an effect on a coding polynucleotide (for example, a gene), refers to a measurable decrease in the cellular level of mRNA transcribed from the coding polynucleotide and/or peptide, polypeptide, or protein product of the coding polynucleotide. In some examples, expression of a coding polynucleotide may be inhibited such that expression is approximately eliminated. “Specific inhibition” refers to the inhibition of a target coding polynucleotide without consequently affecting expression of other coding polynucleotides (e.g., genes) in the cell wherein the specific inhibition is being accomplished.

Insect: As used herein with regard to pests, the term “insect pest” specifically includes pollen beetles.

Isolated: An “isolated” biological component (such as a nucleic acid molecule or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a polynucleotide may be isolated from a chromosome by breaking chemical bonds connecting the polynucleotide to the remaining DNA in the chromosome). Nucleic acid molecules and proteins that have been “isolated” include nucleic acid molecules and proteins purified by standard purification methods. The term also embraces RNA molecules and proteins prepared by recombinant expression in a host cell, as well as chemically-synthesized nucleic acid molecules, proteins, and peptides.

Nucleic acid molecule: As used herein, the term “nucleic acid molecule” may refer to a polymeric form of nucleotides, which may include both sense and anti-sense strands of RNA, cDNA, gDNA, and synthetic forms and mixed polymers of the above. A nucleotide or nucleobase may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. By convention, the nucleotide sequence of a nucleic acid molecule is read from the 5′ to the 3′ end of the molecule. The “complement” of a nucleic acid molecule refers to a polynucleotide having nucleobases that may form base pairs with the nucleobases of the nucleic acid molecule (i.e., A-T/U, and G-C).

Some embodiments include nucleic acids comprising a template DNA that is transcribed into an RNA molecule that comprises a polyribonucleotide that hybridizes to a mRNA molecule. In some examples, the template DNA is the complement of the polynucleotide transcribed into the mRNA molecule, present in the 5′ to 3′ orientation, such that RNA polymerase (which transcribes DNA in the 5′ to 3′ direction) will transcribe the polyribonucleotide from the complement that can hybridize to the mRNA molecule. Unless explicitly stated otherwise, or it is clear to be otherwise from the context, the term “complement” therefore refers to a polynucleotide having nucleobases, from 5′ to 3′, that may form base pairs with the nucleobases of a reference nucleic acid. In some examples, the template DNA is the reverse complement of the polynucleotide transcribed into the mRNA molecule. Thus, unless it is explicitly stated to be otherwise (or it is clear to be otherwise from the context), the “reverse complement” of a polynucleotide refers to the complement in reverse orientation. The foregoing is demonstrated in the following illustration:

| | |
|---|---|
| ATGATGATG | polynucleotide |
| TACTACTAC | “complement” of the polynucleotide |
| CATCATCAT | “reverse complement” of the |
| | polynucleotide |

Some embodiments of the invention include hairpin RNA-forming RNAi molecules. In these RNAi molecules, both a nucleotide sequence of a polynucleotide to be targeted by RNA interference and its reverse complement may be found in the same molecule, such that the single-stranded RNA molecule may “fold over” and hybridize to itself over the region comprising the nucleotide sequence and reverse complement of the nucleotide sequence.

“Nucleic acid molecules” include all polynucleotides, for example: single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), shRNA (small hairpin RNA), mRNA (messenger RNA), miRNA (micro-RNA), hpRNA (hairpin RNA), tRNA (transfer RNAs, whether charged or discharged with a corresponding acylated amino acid), and cRNA (complementary RNA). The term “deoxyribonucleic acid” (DNA) is inclusive of cDNA, gDNA, and DNA-RNA hybrids. The terms “polynucleotide” and “nucleic acid,” and “fragments” thereof will be understood by those in the art as a term that includes both gDNAs, ribosomal RNAs, transfer RNAs, messenger RNAs, operons, and smaller engineered polynucleotides that encode or may be adapted to encode, peptides, polypeptides, or proteins.

Oligonucleotide: An oligonucleotide is a short nucleic acid polymer. Oligonucleotides may be formed by cleavage of longer nucleic acid segments, or by polymerizing individual nucleotide precursors. Automated synthesizers allow the synthesis of oligonucleotides up to several hundred bases in length. Because oligonucleotides may bind to a complementary nucleic acid, they may be used as probes for detecting DNA or RNA. Oligonucleotides composed of DNA (oligodeoxyribonucleotides) may be used in PCR, a technique for the amplification of DNAs. In PCR, the oligonucleotide is typically referred to as a “primer,” which allows a DNA polymerase to extend the oligonucleotide and replicate the complementary strand.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding polynucleotide,” “structural polynucleotide,” or “structural nucleic acid molecule” refers to a polynucleotide that is ultimately translated into a polypeptide, via transcription and mRNA, when placed under the control of appropriate regulatory elements. With respect to RNA, the term “coding polynucleotide” refers to a polynucleotide that is translated into a peptide, polypeptide, or protein. The boundaries of a coding polynucleotide are determined by a translation start codon at the 5′-terminus and a translation stop codon at the 3′-terminus. Coding polynucleotides include, but are not limited to: gDNA; cDNA; EST; and recombinant polynucleotides.

As used herein, “transcribed non-coding polynucleotide” refers to segments of mRNA molecules such as 5′UTR, 3′UTR and intron segments that are not translated into a peptide, polypeptide, or protein. Further, “transcribed non-coding polynucleotide” refers to a nucleic acid that is transcribed into an RNA that functions in the cell, for example, structural RNAs (e.g., ribosomal RNA (rRNA) as exemplified by 5S rRNA, 5.8S rRNA, 16S rRNA, 18 S rRNA, 23 S rRNA, and 28S rRNA, and the like); transfer RNA (tRNA); and snRNAs such as U4, U5, U6, and the like. Transcribed non-coding polynucleotides also include, for example and without limitation, small RNAs (sRNA), which term is often used to describe small bacterial non-coding RNAs; small nucleolar RNAs (snoRNA); microRNAs; small interfering RNAs (siRNA); Piwi-interacting RNAs (piRNA); and long non-coding RNAs. Further still, “transcribed non-coding polynucleotide” refers to a polynucleotide that may natively exist as an intragenic “spacer” in a nucleic acid and which is transcribed into an RNA molecule.

Lethal RNA interference: As used herein, the term “lethal RNA interference” refers to RNA interference that results in death or a reduction in viability of the subject individual to which, for example, a dsRNA, miRNA, siRNA, shRNA, and/or hpRNA is delivered.

Genome: As used herein, the term “genome” refers to chromosomal DNA found within the nucleus of a cell, and also refers to organelle DNA found within subcellular components of the cell. In some embodiments of the invention, a DNA molecule may be introduced into a plant cell, such that the DNA molecule is integrated into the genome of the plant cell. In these and further embodiments, the DNA molecule may be either integrated into the nuclear DNA of the plant cell, or integrated into the DNA of the chloroplast or mitochondrion of the plant cell. The term “genome,” as it applies to bacteria, refers to both the chromosome and plasmids within the bacterial cell. In some embodiments of the invention, a DNA molecule may be introduced into a bacterium such that the DNA molecule is integrated into the genome of the bacterium. In these and further embodiments, the DNA molecule may be either chromosomally-integrated or located as or in a stable plasmid.

Sequence identity: The term “sequence identity” or “identity,” as used herein in the context of two polynucleotides or polypeptides, refers to the residues in the sequences of the two molecules that are the same when aligned for maximum correspondence over a specified comparison window.

As used herein, the term “percentage of sequence identity” may refer to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default BLOSUM62 matrix set to default parameters. Nucleic acids with even greater sequence similarity to the sequences of the reference polynucleotides will show increasing percentage identity when assessed by this method.

Specifically hybridizable/Specifically complementary: As used herein, the terms “Specifically hybridizable” and “Specifically complementary” are terms that indicate a sufficient degree of complementarity such that stable and specific binding occurs between the nucleic acid molecule and a target nucleic acid molecule. Hybridization between two nucleic acid molecules involves the formation of an anti-parallel alignment between the nucleobases of the two nucleic acid molecules. The two molecules are then able to form hydrogen bonds with corresponding bases on the opposite strand to form a duplex molecule that, if it is sufficiently stable, is detectable using methods well known in the art. A polynucleotide need not be 100% complementary to its target nucleic acid to be specifically hybridizable. However, the amount of complementarity that must exist for hybridization to be specific is a function of the hybridization conditions used.

Hybridization conditions resulting in particular degrees of stringency will vary depending upon the nature of the hybridization method of choice and the composition and length of the hybridizing nucleic acids. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N.Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology-Hybridization with Nucleic Acid Probes, Part I, Chapter 2, Elsevier, N Y, 1993; and Ausubel et al., Eds., Current Protocols in Molecular Biology, Chapter 2, Greene Publishing and Wiley-Interscience, N Y, 1995.

As used herein, “stringent conditions” encompass conditions under which hybridization will only occur if there is less than 20% mismatch between the sequence of the hybridization molecule and a homologous polynucleotide within the target nucleic acid molecule. “Stringent conditions” include further particular levels of stringency. Thus, as used herein, “moderate stringency” conditions are those under which molecules with more than 20% sequence mismatch will not hybridize; conditions of “high stringency” are those under which sequences with more than 10% mismatch will not hybridize; and conditions of “very high stringency” are those under which sequences with more than 5% mismatch will not hybridize.

The following are representative, non-limiting hybridization conditions.

High Stringency condition (detects polynucleotides that share at least 90% sequence identity): Hybridization in 5×SSC buffer at 65° C. for 16 hours; wash twice in 2×SSC buffer at room temperature for 15 minutes each; and wash twice in 0.5×SSC buffer at 65° C. for 20 minutes each.

Moderate Stringency condition (detects polynucleotides that share at least 80% sequence identity): Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.

Non-stringent control condition (polynucleotides that share at least 50% sequence identity will hybridize): Hybridization in 6×SSC buffer at room temperature to 55° C. for 16-20 hours; wash at least twice in 2×-3×SSC buffer at room temperature to 55° C. for 20-30 minutes each.

As used herein, the term “substantially homologous,” “substantially identical,” or “substantial homology,” with regard to a reference polynucleotide or polyribonucleotide, refers to a polynucleotide or polyribonucleotide having contiguous nucleobases that hybridize under stringent conditions to a oligonucleotide consisting of the nucleotide sequence of the reference polynucleotide or polyribonucleotide. For example, polynucleotides that are substantially homologous to a reference polynucleotide of any of SEQ ID NOs:2-4 are those polynucleotides that hybridize under stringent conditions (e.g., the Moderate Stringency conditions set forth, supra) to an oligonucleotide consisting of the nucleotide sequence of the reference polynucleotide. Substantially homologous or substantially identical polynucleotides may have at least 80% sequence identity. For example, substantially identical polynucleotides may have from about 80% to 100% sequence identity, such as 79%; 80%; about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial identity is closely related to specific hybridization. For example, a nucleic acid molecule is specifically hybridizable when there is a sufficient degree of complementarity to avoid non-specific binding of the nucleic acid to non-target polynucleotides under conditions where specific binding is desired, for example, under stringent hybridization conditions.

As used herein, the term “ortholog” refers to a gene in two or more species that has evolved from a common ancestral nucleic acid, and may retain the same function in the two or more species.

As used herein, two polynucleotides are said to exhibit “complete complementarity” when every nucleotide of a polynucleotide read in the 5′ to 3′ direction is complementary to every nucleotide of the other polynucleotide when read in the 5′ to 3′ direction. Similarly, a polynucleotide that is completely reverse complementary to a reference polynucleotide will exhibit a nucleotide sequence where every nucleotide of the polynucleotide read in the 5′ to 3′ direction is complementary to every nucleotide of the reference polynucleotide when read in the 3′ to 5′ direction. These terms and descriptions are well defined in the art and are easily understood by those of ordinary skill in the art.

Operably linked: A first polynucleotide is operably linked with a second polynucleotide when the first polynucleotide is in a functional relationship with the second polynucleotide. When recombinantly produced, operably linked polynucleotides are generally contiguous, and, where necessary to join two protein-coding regions, in the same reading frame (e.g., in a translationally fused ORF). However, nucleic acids need not be contiguous to be operably linked.

The term, “operably linked,” when used in reference to a regulatory genetic element and a coding polynucleotide, means that the regulatory element affects the expression of the linked coding polynucleotide. “Regulatory elements,” or “control elements,” refer to polynucleotides that influence the timing and level/amount of transcription, RNA processing or stability, or translation of the associated coding polynucleotide. Regulatory elements may include promoters; translation leaders; introns; enhancers; stem-loop structures; repressor binding polynucleotides; polynucleotides with a termination sequence; polynucleotides with a polyadenylation recognition sequence; etc. Particular regulatory elements may be located upstream and/or downstream of a coding polynucleotide operably linked thereto. Also, particular regulatory elements operably linked to a coding polynucleotide may be located on the associated complementary strand of a double-stranded nucleic acid molecule.

Promoter: As used herein, the term “promoter” refers to a region of DNA that may be upstream from the start of transcription, and that may be involved in recognition and binding of RNA polymerase and other proteins to initiate transcription. A promoter may be operably linked to a coding polynucleotide for expression in a cell, or a promoter may be operably linked to a polynucleotide encoding a signal peptide which may be operably linked to a coding polynucleotide for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissues are referred to as “tissue-specific”. A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most tissue or cell types.

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that respond to copper; Int gene from maize that responds to benzenesulfonamide herbicide safeners; Tet repressor from Tn10; and the inducible promoter from a steroid hormone gene, the transcriptional activity of which may be induced by a glucocorticosteroid hormone (Schena et al. (1991) Proc. Natl. Acad. Sci. USA 88:0421).

Exemplary constitutive promoters include, but are not limited to: Promoters from plant viruses, such as the 35S promoter from Cauliflower Mosaic Virus (CaMV); promoters from rice actin genes; ubiquitin promoters; pEMU; MAS; maize H3 histone promoter; and the ALS promoter, XbaI/NcoI fragment 5′ to the Brassica napus ALS3 structural gene (or a polynucleotide similar to said XbaI/NcoI fragment) (International PCT Publication No. WO96/30530).

Additionally, any tissue-specific or tissue-preferred promoter may be utilized in some embodiments of the invention. Plants transformed with a nucleic acid molecule comprising a coding polynucleotide operably linked to a tissue-specific promoter may produce the product of the coding polynucleotide exclusively, or preferentially, in a specific tissue. Exemplary tissue-specific or tissue-preferred promoters include, but are not limited to: A seed-preferred promoter, such as that from the phaseolin gene; a leaf-specific and light-induced promoter such as that from cab or rubisco; an anther-specific promoter such as that from LAT52; a pollen-specific promoter such as that from Zm13; and a microspore-preferred promoter such as that from apg.

Rapeseed/Oilseed Rape plant: As used herein, the term “rapeseed” or “oilseed rape” refers to a plant of the genus, Brassica; for example, a canola plant of the species Brassica napus.

Transformation: As used herein, the term “transformation” or “transduction” refers to the transfer of one or more nucleic acid molecule(s) into a cell. A cell is “transformed” by a nucleic acid molecule transduced into the cell when the nucleic acid molecule becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; and microprojectile bombardment (Klein et al. (1987) Nature 327:70).

Transgene: An exogenous polynucleotide. In some examples, a transgene may be a DNA that encodes one or both strand(s) of an RNA capable of forming a dsRNA molecule that comprises a nucleotide sequence that is complementary to a nucleic acid molecule found in pollen beetle. In further examples, a transgene may be a gene (e.g., a herbicide-tolerance gene, a gene encoding an industrially or pharmaceutically useful compound, or a gene encoding a desirable agricultural trait). In these and other examples, a transgene may contain regulatory elements operably linked to a coding polynucleotide of the transgene (e.g., a promoter).

Vector: A nucleic acid molecule as introduced into a cell, for example, to produce a transformed cell. A vector may include genetic elements that permit it to replicate in the host cell, such as an origin of replication. Examples of vectors include, but are not limited to: a plasmid; cosmid; bacteriophage; or virus that carries exogenous DNA into a cell. A vector may also include one or more genes, including ones that produce antisense molecules, and/or selectable marker genes and other genetic elements known in the art. A vector may transduce, transform, or infect a cell, thereby causing the cell to express the nucleic acid molecules and/or proteins encoded by the vector. A vector optionally includes materials to aid in achieving entry of the nucleic acid molecule into the cell (e.g., a liposome, protein coating, etc.).

Yield: A stabilized yield of about 100% or greater relative to the yield of check varieties in the same growing location growing at the same time and under the same conditions. In particular embodiments, “improved yield” or “improving yield” means a cultivar having a stabilized yield of 105% or greater relative to the yield of check varieties in the same growing location containing significant densities of the insect pests that are injurious to that crop growing at the same time and under the same conditions, which are targeted by the compositions and methods herein.

Unless specifically indicated or implied, the terms “a,” “an,” and “the” signify “at least one,” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin's Genes X, Jones & Bartlett Publishers, 2009 (ISBN 10 0763766321); Krebs et al. (eds.), The Encyclopedia of Molecular Biology, Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8). All percentages are by weight and all solvent mixture proportions are by volume unless otherwise noted. All temperatures are in degrees Celsius.

### IV. Nucleic Acid Molecules Comprising an Insect Pest Sequence

A. Overview

Described herein are nucleic acid molecules useful for the control of insect pests. In some examples, the insect pest is Meligethes aeneus Fabricius. Described nucleic acid molecules include target polynucleotides (e.g., native genes, and non-coding polynucleotides), dsRNAs, siRNAs, shRNAs, hpRNAs, and miRNAs. For example, dsRNA, siRNA, miRNA, shRNA, and/or hpRNA molecules are described in some embodiments that may be specifically complementary to all or part of one or more native nucleic acids in an insect pest. In these and further embodiments, the native nucleic acid(s) may be one or more target gene(s), the product of which may be, for example and without limitation: involved in a metabolic process or involved in larval development. Nucleic acid molecules described herein, when introduced into a cell comprising at least one native nucleic acid(s) to which the nucleic acid molecules are specifically complementary, may initiate RNAi in the cell, and consequently reduce or eliminate expression of the native nucleic acid(s). In some examples, reduction or elimination of the expression of a target gene by a nucleic acid molecule specifically complementary thereto may result in reduction or cessation of growth, development, and/or feeding in the insect pest.

In some embodiments, at least one target gene in an insect pest may be selected, wherein the target gene comprises a prp19 polynucleotide. In particular examples, a target gene comprising a prp19 polynucleotide is selected, wherein the target gene is the PB prp19 gene comprising SEQ ID NOs:2-3 or a Meligethes gene comprising SEQ ID NO:4.

In some embodiments, a target gene may be a nucleic acid molecule comprising a polynucleotide that can be reverse translated in silico to a polypeptide comprising a contiguous amino acid sequence that is at least about 85% identical (e.g., at least 84%, 85%, about 90%, about 95%, about 96%, about 97%, about 98%, about 99%, about 100%, or 100% identical) to the amino acid sequence of a protein product of a prp19 polynucleotide. In particular examples, a target gene is a nucleic acid molecule comprising a polynucleotide that can be reverse translated in silico to a polypeptide comprising a contiguous amino acid sequence that is at least about 85% identical, about 90% identical, about 95% identical, about 96% identical, about 97% identical, about 98% identical, about 99% identical, about 100% identical, or 100% identical to an amino acid sequence selected from the group consisting of SEQ ID NO:5, the PB PRP19 comprising SEQ ID NOs:6-7, and peptide fragments of the foregoing.

Provided according to the invention are DNAs, the expression of which results in an RNA molecule comprising a polynucleotide that is specifically complementary to all or part of a native RNA molecule that is encoded by a coding polynucleotide in pollen beetle. In some embodiments, after ingestion of the expressed RNA molecule by an insect pest, down-regulation of the coding polynucleotide in cells of the pest may be obtained. In particular embodiments, down-regulation of the coding sequence in cells of the insect pest may result in a deleterious effect on the growth development, and/or survival of the pest.

In some embodiments, target polynucleotides include transcribed non-coding RNAs, such as 5′UTRs; 3′UTRs; spliced leaders; introns; outrons (e.g., 5′UTR RNA subsequently modified in trans splicing); donatrons (e.g., non-coding RNA required to provide donor sequences for trans splicing); and other non-coding transcribed RNA of target insect pest genes. Such polynucleotides may be derived from both mono-cistronic and poly-cistronic genes.

Thus, also described herein in connection with some embodiments are iRNA molecules (e.g., dsRNAs, siRNAs, miRNAs, shRNAs, and hpRNAs) that comprise at least one nucleotide sequence that is specifically complementary to all or part of a target polynucleotide in pollen beetle. In some embodiments, an iRNA molecule may comprise nucleotide sequence(s) that are complementary to all or part of a plurality of target polynucleotides; for example, 2, 3, 4, 5, 6, 7, 8, 9, 10, or more target polynucleotides. In particular embodiments, an iRNA molecule may be produced in vitro or in vivo by a genetically-modified organism, such as a plant or bacterium. Also disclosed are cDNAs that may be used for the production of dsRNA molecules, siRNA molecules, miRNA molecules, shRNA molecules, and/or hpRNA molecules that are specifically complementary to all or part of a target polynucleotide in an insect pest. Further described are recombinant DNA constructs for use in achieving stable transformation of particular host targets. Transformed host targets may express effective levels of dsRNA, siRNA, miRNA, shRNA, and/or hpRNA molecules from the recombinant DNA constructs. Therefore, also described is a plant transformation vector comprising at least one polynucleotide operably linked to a heterologous promoter functional in a plant cell, wherein expression of the polynucleotide(s) results in an RNA molecule comprising at least one contiguous nucleotide sequence that is specifically complementary to all or part of a target polynucleotide in an insect pest.

In particular examples, nucleic acid molecules useful for the control of insect pests comprise: SEQ ID NO:1; the native coding polynucleotide isolated from pollen beetle comprising SEQ ID NOs:2-3; all or part of a native prp19 polynucleotide isolated from Meligethes comprising any of SEQ ID NOs:2-4); DNAs that when expressed result in an RNA molecule comprising a polyribonucleotide that is specifically complementary or reverse complementary to all or part of a native RNA molecule that is encoded by Meligethes prp19; iRNA molecules (e.g., dsRNAs, siRNAs, miRNAs, shRNAs, and hpRNAs) that comprise at least one polyribonucleotide that is specifically complementary or reverse complementary to all or part of Meligethes prp19; cDNAs that may be used for the production of dsRNA molecules, siRNA molecules, miRNA molecules, shRNA molecules, and/or hpRNA molecules that are specifically complementary or reverse complementary to all or part of Meligethes prp19; and/or recombinant DNA constructs for use in achieving stable transformation of particular host targets, wherein a transformed host target comprises one or more of the foregoing polynucleotides.

B. Nucleic Acid Molecules

The present invention provides, inter alia, iRNA (e.g., dsRNA, siRNA, miRNA, shRNA, and hpRNA) molecules that inhibit target gene expression in a cell, tissue, or organ of an insect pest; and DNA molecules capable of being expressed as an iRNA molecule in a cell or microorganism to inhibit target gene expression in a cell, tissue, or organ of an insect pest.

Some embodiments of the invention provide an isolated or recombinant nucleic acid molecule characterized by a polynucleotide comprising at least one (e.g., one, two, three, or more) nucleotide sequence(s) selected from the group consisting of: SEQ ID NO:1; the complement or reverse complement of SEQ ID NO:1; the PB prp19 polynucleotide comprising SEQ ID NOs:2-3, the complement or reverse complement of the PB prp19 polynucleotide comprising SEQ ID NOs:2-3; a fragment of at least 15 (e.g, at least 19) contiguous nucleotides of the PB prp19 polynucleotide comprising SEQ ID NOs:2-3 (e.g., SEQ ID NO:4); the complement or reverse complement of a fragment of at least 15 contiguous nucleotides of the PB prp19 polynucleotide comprising SEQ ID NOs:2-3; a native coding polynucleotide of a Meligethes organism (e.g., PB) comprising SEQ ID NO:4; the complement or reverse complement of a native coding polynucleotide of a Meligethes organism comprising SEQ ID NO:4; a fragment of at least 15 contiguous nucleotides of a native coding polynucleotide of a Meligethes organism comprising SEQ ID NO:4; and the complement or reverse complement of a fragment of at least 15 contiguous nucleotides of a native coding polynucleotide of a Meligethes organism comprising SEQ ID NO:4.

In particular embodiments, contact with or uptake by an insect pest of an iRNA transcribed from the foregoing polynucleotides inhibits the growth, development, and/or feeding of the pest. In some embodiments, contact with or uptake by the insect occurs via feeding on plant material comprising the iRNA. In some embodiments, contact with or uptake by the insect occurs via spraying of a plant comprising the insect with a composition comprising the iRNA.

In some embodiments, a nucleic acid molecule of the invention is an iRNA molecule characterized by a polyribonucleotide comprising at least one (e.g., one, two, three, or more) nucleotide sequence(s) selected from the group consisting of: SEQ ID NO:12; the complement or reverse complement of SEQ ID NO:12; SEQ ID NO:13; the complement or reverse complement of SEQ ID NO:13; SEQ ID NO:14; the complement or reverse complement of SEQ ID NO:14; SEQ ID NO:15; the complement or reverse complement of SEQ ID NO:15; a fragment of at least 15 (e.g., at least 19) contiguous nucleotides of any of SEQ ID NOs:13-15 (e.g., any of SEQ ID NOs:17-31); the complement or reverse complement of a fragment of at least 15 contiguous nucleotides of any of SEQ ID NOs:13-15; a native polyribonucleotide transcribed in pollen beetle comprising SEQ ID NOs:13-14; the complement or reverse complement of a native polyribonucleotide transcribed in pollen beetle comprising SEQ ID NOs:13-14; a fragment of at least 15 contiguous nucleotides of a native polyribonucleotide transcribed in pollen beetle comprising SEQ ID NOs:13-14; the complement or reverse complement of a fragment of at least 15 contiguous nucleotides of a native polyribonucleotide transcribed in pollen beetle comprising SEQ ID NOs:13-14; a native polyribonucleotide transcribed in a Meligethes organism comprising SEQ ID NO:15; the complement or reverse complement of a native polyribonucleotide transcribed in a Meligethes organism comprising SEQ ID NO:15; a fragment of at least 15 contiguous nucleotides of a native polyribonucleotide transcribed in a Meligethes organism comprising SEQ ID NO:15; and the complement or reverse complement of a fragment of at least 15 contiguous nucleotides of a native polyribonucleotide transcribed in a Meligethes organism comprising SEQ ID NO:15.

In particular embodiments, contact with or uptake by an insect pest of the iRNA molecule inhibits the growth, development, and/or feeding of the pest. In some embodiments, contact with or uptake by the insect occurs via feeding on plant material or bait comprising the iRNA. In some embodiments, contact with or uptake by the insect pest occurs via spraying of a plant comprising the insect with a composition comprising the iRNA.

In certain embodiments, dsRNA molecules provided by the invention comprise polyribonucleotides comprising at least one nucleotide sequence that is complementary (or reverse complementary) to a transcript from a target gene comprising any of SEQ ID NOs:1-4, and fragments thereof, the inhibition of which target gene in an insect pest results in the reduction or removal of a polypeptide or polynucleotide agent that is essential for the pest's growth, development, or other biological function. A selected target polynucleotide may exhibit from about 80% to about 100% sequence identity to a reference polynucleotide selected from the group consisting of any of SEQ ID NOs:1-4; a contiguous fragment of the PB prp19 gene comprising SEQ ID NOs:2-3; a contiguous fragment of one or more of SEQ ID NOs:2-4; and the complements and reverse complements of the foregoing. For example, a selected polynucleotide may exhibit 79%; 80%; about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; or about 100% sequence identity to any of the foregoing reference polynucleotides.

In some examples, a dsRNA molecule is transcribed from a polynucleotide containing a sense nucleotide sequence that is substantially identical or identical to a contiguous fragment of the PB prp19 gene comprising SEQ ID NOs:2-3 (e.g., SEQ ID NO:4); an antisense nucleotide sequence that is at least substantially the reverse complement of the sense nucleotide sequence; and an intervening nucleotide sequence positioned between the sense and the antisense sequences, such that the sense and antisense polyribonucleotides transcribed from the respective sense and antisense nucleotide sequences hybridize to form a “stem” structure in the dsRNA, and polyribonucleotide transcribed from the intervening sequence forms a “loop.” Such a dsRNA molecule may be referred to as a hairpin RNA (hpRNA) molecule. An example of such a hpRNA molecule is SEQ ID NO:16, encoded by the polynucleotide of SEQ ID NO:11, which contains the sense nucleotide sequence of SEQ ID NO:4.

In some embodiments, a polynucleotide capable of being expressed as an iRNA molecule in a cell or microorganism to inhibit target gene expression may comprise a single nucleotide sequence that is specifically complementary or reverse complementary to all or part of a native polynucleotide found in pollen beetle, or the polynucleotide can be constructed as a chimera, comprising a plurality of such specifically complementary or reverse complementary nucleotide sequences.

In some embodiments, a polynucleotide may comprise a first and a second nucleotide sequence separated by a “spacer.” A spacer may be a region comprising any sequence of nucleotides that facilitates secondary structure formation between the first and second polynucleotides or their transcription products, where this is desired. In one embodiment, the spacer is part of a sense or antisense coding polyribonucleotide for mRNA. The spacer may alternatively comprise any combination of nucleotides or homologues thereof that are capable of being linked covalently in a nucleic acid molecule. In some examples, the spacer may be an intron.

For example, in some embodiments, a DNA molecule may comprise polynucleotide(s) encoding one or more different iRNA molecules, wherein each of the different iRNA molecules comprises a first nucleotide sequence and a second nucleotide sequence, wherein the first and second nucleotide sequences are complementary to each other. The first and second nucleotide sequences may be connected within the iRNA molecule by a spacer. The spacer may constitute part of the first nucleotide sequence or the second nucleotide sequence. Expression of an iRNA molecule comprising the first and second nucleotide sequences may lead to the formation of a hpRNA molecule, by specific intramolecular base-pairing of the first and second nucleotide sequences. The first nucleotide sequence or the second nucleotide sequence may be substantially identical to the polyribonucleotide encoded by a polynucleotide (e.g., a target gene, fragment of a target gene, or transcribed non-coding polynucleotide) native to an insect pest, or the complement or reverse complement thereof.

dsRNA nucleic acid molecules comprise double strands of polymerized ribonucleotides, and may include modifications to either the phosphate-sugar backbone or the nucleoside. Modifications in RNA structure may be tailored to allow specific inhibition. In one embodiment, dsRNA molecules may be modified through an ubiquitous enzymatic process so that siRNA molecules may be generated. This enzymatic process may utilize an RNase III enzyme, such as DICER in eukaryotes, either in vitro or in vivo. See Elbashir et al. (2001) Nature 411:494-8; and Hamilton and Baulcombe (1999) Science 286(5441):950-2. DICER or functionally-equivalent RNase III enzymes cleave larger dsRNA strands and/or hpRNA molecules into smaller oligonucleotides (e.g., siRNAs), each of which is about 19-25 nucleotides in length. The siRNA molecules produced by these enzymes have 2 to 3 nucleotide 3′ overhangs, and 5′ phosphate and 3′ hydroxyl termini. The siRNA molecules generated by RNase III enzymes are unwound and separated into single-stranded RNA in the cell. The siRNA molecules then specifically hybridize with RNAs transcribed from a target gene, and both RNA molecules are subsequently degraded by an inherent cellular RNA-degrading mechanism. This process may result in the effective degradation or removal of the RNA encoded by the target gene in the target organism. The outcome is the post-transcriptional silencing of the targeted gene. In some embodiments, siRNA molecules produced by endogenous RNase III enzymes from heterologous nucleic acid molecules may efficiently mediate the down-regulation of target genes in insect pests.

In some embodiments, a nucleic acid molecule may include at least one non-naturally occurring polynucleotide that can be transcribed into a single-stranded RNA molecule capable of forming a dsRNA molecule in vivo through intermolecular hybridization. Such dsRNAs typically self-assemble, and can be provided in the nutrition source of an insect pest to achieve the post-transcriptional inhibition of a target gene. In these and further embodiments, a nucleic acid molecule may comprise two different non-naturally occurring polynucleotides, each of which comprises at least one nucleotide sequence that is specifically complementary or reverse complementary to a different target gene in an insect pest. When such a nucleic acid molecule is provided as a dsRNA molecule to, for example, a pollen beetle, the dsRNA molecule inhibits the expression of at least two different target genes in the pest.

C. Obtaining Nucleic Acid Molecules

A variety of polynucleotides in insect pests may be used as targets for the design of nucleic acid molecules, such as iRNAs and DNA molecules encoding iRNAs. Selection of native polynucleotides is not, however, a straight-forward process. For example, only a small number of native polynucleotides in an insect pest will be effective targets. It cannot be predicted with certainty whether a particular native polynucleotide can be effectively down-regulated by nucleic acid molecules of the invention, or whether down-regulation of a particular native polynucleotide will have a detrimental effect on the growth, development, and/or survival of an insect pest. The vast majority of native insect pest polynucleotides, such as ESTs isolated therefrom (for example, the Western Corn Rootworm polynucleotides listed in U.S. Pat. No. 7,612,194), do not have a detrimental effect on the growth, development, and/or survival of the pest. Neither is it predictable which of the native polynucleotides that may have a detrimental effect on an insect pest are able to be used in recombinant techniques for expressing nucleic acid molecules complementary to such native polynucleotides in a host plant and providing the detrimental effect on the pest upon feeding without causing harm to the host plant.

In some embodiments, nucleic acid molecules (e.g., dsRNA molecules to be provided in the host plant of an insect pest) target cDNAs that encode proteins or parts of proteins essential for pest development and/or survival, such as polypeptides involved in metabolic or catabolic biochemical pathways, cell division, energy metabolism, digestion, host plant recognition, and the like. As described herein, ingestion of compositions by a target pest organism containing one or more dsRNAs, at least one segment of which is specifically complementary to at least a substantially identical segment of RNA produced in the cells of the target pest organism, can result in the death or other inhibition of the target. A polynucleotide derived from a native insect pest gene can be used to construct plant cells resistant to infestation by the pests. The host plant (e.g., B. napus) of an insect pest, for example, can be transformed to contain one or more polynucleotides derived from pollen beetle as provided herein. The polynucleotide transformed into the host may encode one or more RNAs that form into a dsRNA structure in the cells or biological fluids within the transformed host, thus making the dsRNA available if/when the pest forms a nutritional relationship with the transgenic host. This may result in the suppression of expression of one or more genes in the cells of the pest, and ultimately death or inhibition of its growth or development.

In particular embodiments, a gene is targeted that is essentially involved in the growth and development of an insect pest. Other target genes for use in the present invention may include, for example, those that play important roles in pest viability, movement, migration, growth, development, infectivity, and establishment of feeding sites. A target gene may therefore be a housekeeping gene or a transcription factor.

In some embodiments, the invention provides methods for obtaining a nucleic acid molecule comprising a polynucleotide for producing an iRNA (e.g., dsRNA, siRNA, miRNA, shRNA, and hpRNA) molecule. One such embodiment comprises: (a) analyzing one or more target gene(s) for their expression, function, and phenotype upon dsRNA-mediated gene suppression in an insect pest (e.g., pollen beetle); (b) probing a cDNA or gDNA library with a probe comprising all or a portion of a polynucleotide or a homolog thereof from a targeted pest that displays an altered (e.g., reduced) growth or development phenotype in a dsRNA-mediated suppression analysis; (c) identifying a DNA clone that specifically hybridizes with the probe; (d) isolating the DNA clone identified in step (b); (e) sequencing the cDNA or gDNA fragment that comprises the clone isolated in step (d), wherein the sequenced nucleic acid molecule comprises all or a substantial portion of the RNA or a homolog thereof; and (f) chemically synthesizing all or a substantial portion of a gene, or an siRNA, miRNA, hpRNA, mRNA, shRNA, or dsRNA.

In further embodiments, a method for obtaining a nucleic acid fragment comprising a polynucleotide for producing a substantial portion of an iRNA molecule includes: (a) synthesizing first and second oligonucleotide primers specifically complementary to a portion of a native polynucleotide from a targeted insect pest; and (b) amplifying a cDNA or gDNA insert present in a cloning vector using the first and second oligonucleotide primers of step (a), wherein the amplified nucleic acid molecule comprises a substantial portion of the iRNA molecule.

Polynucleotides can be isolated, amplified, or produced by a number of approaches. For example, an iRNA molecule may be obtained by PCR amplification of a target polynucleotide (e.g., a target gene, fragment of a target gene, and a target transcribed non-coding polynucleotide) derived from a gDNA or cDNA library, or portions thereof. DNA or RNA may be extracted from a target organism, and nucleic acid libraries may be prepared therefrom using methods known to those ordinarily skilled in the art. gDNA or cDNA libraries generated from a target organism may be used for PCR amplification and sequencing of target genes. A confirmed PCR product may be used as a template for in vitro transcription to generate sense and antisense RNA with minimal promoters. Alternatively, nucleic acid molecules may be synthesized by any of a number of techniques (See, e.g., Ozaki et al. (1992) Nucleic Acids Research, 20: 5205-5214; and Agrawal et al. (1990) Nucleic Acids Research, 18: 5419-5423), including use of an automated DNA synthesizer (for example, a P. E. Biosystems, Inc. (Foster City, Calif.) model 392 or 394 DNA/RNA Synthesizer), using standard chemistries, such as phosphoramidite chemistry. See, e.g., Beaucage et al. (1992) Tetrahedron, 48: 2223-2311; U.S. Pat. Nos. 4,980,460, 4,725,677, 4,415,732, 4,458,066, and 4,973,679. Alternative chemistries resulting in non-natural backbone groups, such as phosphorothioate, phosphoramidate, and the like, can also be employed.

An RNA, dsRNA, siRNA, miRNA, shRNA, or hpRNA molecule of the present invention may be produced chemically or enzymatically by one skilled in the art through manual or automated reactions, or in vivo in a cell comprising a nucleic acid molecule comprising a polynucleotide encoding the RNA, dsRNA, siRNA, miRNA, shRNA, or hpRNA molecule. RNA may also be produced by partial or total organic synthesis; any modified polyribonucleotide can be introduced by in vitro enzymatic or organic synthesis. An RNA molecule may be synthesized by a cellular RNA polymerase or a bacteriophage RNA polymerase (e.g., T3 RNA polymerase, T7 RNA polymerase, and SP6 RNA polymerase). Expression constructs useful for the cloning and expression of polynucleotides are known in the art. See, e.g., International PCT Publication No. WO97/32016; and U.S. Pat. Nos. 5,593,874, 5,698,425, 5,712,135, 5,789,214, and 5,804,693. RNA molecules that are synthesized chemically or by in vitro enzymatic synthesis may be purified prior to introduction into a cell. For example, RNA molecules can be purified from a mixture by extraction with a solvent or resin, precipitation, electrophoresis, chromatography, or a combination thereof. Alternatively, RNA molecules that are synthesized chemically or by in vitro enzymatic synthesis may be used with no or a minimum of purification, for example, to avoid losses due to sample processing. The RNA molecules may be dried for storage or dissolved in an aqueous solution. The solution may contain buffers or salts to promote annealing, and/or stabilization of dsRNA molecule duplex strands.

In embodiments, a dsRNA molecule may be formed by a single self-complementary RNA strand or from two complementary RNA strands. dsRNA molecules may be synthesized either in vivo or in vitro. An endogenous RNA polymerase of a cell may mediate transcription of the one or two RNA strands in vivo, or cloned RNA polymerase may be used to mediate transcription in vivo or in vitro. An endogenous enzyme of a cell may post-transcriptionally process the dsRNA into, for example, miRNA and/or siRNA molecules. Post-transcriptional inhibition of a target gene in an insect pest may be host-targeted by specific transcription in an organ, tissue, or cell type of the host (e.g., by using a tissue-specific promoter); stimulation of an environmental condition in the host (e.g., by using an inducible promoter that is responsive to infection, stress, temperature, and/or chemical inducers); and/or engineering transcription at a developmental stage or age of the host (e.g., by using a developmental stage-specific promoter). RNA strands that form a dsRNA molecule, whether transcribed in vitro or in vivo, may or may not be polyadenylated, and may or may not be capable of being translated into a polypeptide by a cell's translational apparatus.

D. Recombinant Vectors and Host Cell Transformation

In some embodiments, the invention also provides a DNA molecule for introduction into a cell (e.g., a bacterial cell, a yeast cell, or a plant cell), wherein the DNA molecule comprises a polynucleotide that, upon expression to RNA and ingestion by an insect pest, achieves suppression of a target gene in a cell, tissue, or organ of the pest. Thus, some embodiments provide a recombinant nucleic acid molecule comprising a polynucleotide capable of being expressed as an iRNA (e.g., dsRNA, siRNA, miRNA, shRNA, and hpRNA) molecule in a plant cell to inhibit target gene expression in an insect pest. In order to initiate or enhance expression, such recombinant nucleic acid molecules may comprise one or more regulatory elements, which regulatory elements may be operably linked to the polynucleotide capable of being expressed as an iRNA. Methods to express a gene suppression molecule in plants are known, and may be used to express a polynucleotide of the present invention. See, e.g., International PCT Publication No. WO06/073727; and U.S. Patent Publication No. 2006/0200878 A1)

In specific embodiments, a recombinant DNA molecule of the invention may comprise a polynucleotide encoding an RNA that may form a dsRNA molecule. Such recombinant DNA molecules may encode RNAs that may form dsRNA molecules capable of inhibiting the expression of endogenous target gene(s) in an insect pest cell upon ingestion. In many embodiments, a transcribed RNA may form a dsRNA molecule that may be provided in a stabilized form; e.g., as a hairpin and stem-and-loop structure.

In some embodiments, one strand of a dsRNA molecule may be formed by transcription from a polynucleotide comprising a nucleotide sequence that is substantially identical to a any of SEQ ID NO:1; the complement or reverse complement of SEQ ID NO:1; the PB prp19 polynucleotide comprising SEQ ID NOs:2-3; the complement or reverse complement of the PB prp19 polynucleotide comprising SEQ ID NOs:2-3; a fragment of at least 15 (e.g., at least 19) contiguous nucleotides of the PB prp19 polynucleotide comprising SEQ ID NOs:2-3 (e.g., SEQ ID NO:4); the complement or reverse complement of a fragment of at least 15 contiguous nucleotides of of the PB prp19 polynucleotide comprising SEQ ID NOs:2-3; a native coding polynucleotide of a Meligethes organism comprising any of any of SEQ ID NOs:2-4; the complement or reverse complement of a native coding polynucleotide of a Meligethes organism comprising any of SEQ ID NOs:2-4; a fragment of at least 15 contiguous nucleotides of a native coding polynucleotide of a Meligethes organism comprising any of SEQ ID NOs:2-4; and the complement or reverse complement of a fragment of at least 15 contiguous nucleotides of a native coding polynucleotide of a Meligethes organism comprising any of SEQ ID NOs:2-4.

In some embodiments, one strand of a dsRNA molecule may be formed by transcription from a polynucleotide that is substantially identical to a polynucleotide selected from the group consisting of SEQ ID NO:4; the complement of SEQ ID NO:4; the reverse complement of SEQ ID NO:4; fragments of at least 15 (e.g., at least 19) contiguous nucleotides of SEQ ID NO:4; the complements of fragments of at least 15 contiguous nucleotides of SEQ ID NO:4; and the reverse complements of fragments of at least 15 contiguous nucletoides of SEQ ID NO:4.

In particular embodiments, a recombinant DNA molecule encoding an RNA that may form a dsRNA molecule may comprise a coding polynucleotide wherein at least two nucleotide sequences are arranged such that one nucleotide sequence is in a sense orientation, and the other nucleotide sequence is in an antisense orientation, relative to at least one promoter, wherein the sense nucleotide sequence and the antisense nucleotide sequence are linked or connected by a spacer of, for example, from about 100 to about 1000 nucleotides. The spacer may form a loop between the sense and antisense nucleotide sequences. The sense nucleotide sequence sequence may be substantially identical to a target gene (e.g., a prp19 gene comprising SEQ ID NOs:2-3) or a fragment thereof. In some embodiments, however, a recombinant DNA molecule may encode an RNA that may form a dsRNA molecule without a spacer. In embodiments, a sense nucleotide sequence and an antisense nucleotide sequence of a polynucleotide encoding a dsRNA molecule may be different lengths.

Polynucleotides identified as having a deleterious effect on an insect pest or a plant-protective effect with regard to the pest may be readily incorporated into expressed dsRNA molecules through the creation of appropriate expression cassettes in a recombinant nucleic acid molecule of the invention. For example, such polynucleotides may be expressed as a hairpin with stem and loop structure by taking a first nucleotide sequence corresponding to a target gene polynucleotide (e.g., a prp19 gene comprising SEQ ID NOs:2-3, and fragments of the foregoing); linking this nucleotide sequence to a second spacer nucleotide sequence that is not homologous or complementary to the first nucleotide sequence; and linking this to a third nucleotide sequence, wherein at least a portion of the third nucleotide sequence is substantially the reverse complement of the first nucleotide sequence. The transcript of such a polynucleotide forms a stem-and-loop structure by intramolecular base-pairing of the first nucleotide sequence with the third nucleotide sequence, wherein the loop structure forms from the transcript of the second nucleotide sequence. See, e.g., U.S. Patent Publication Nos. 2002/0048814 and 2003/0018993; and International PCT Publication Nos. WO94/01550 and WO98/05770. A dsRNA molecule may be generated, for example, in the form of a double-stranded structure such as a stem-loop structure (e.g., hairpin), whereby production of miRNA or siRNA targeted for a native insect pest polynucleotide is enhanced by co-expression of a fragment of the targeted gene, for instance on an additional plant expressible cassette, that leads to enhanced siRNA production, or reduces methylation to prevent transcriptional gene silencing of a promoter operably linked to the polynucleotide encoding the dsRNA molecule.

Certain embodiments of the invention include introduction of a recombinant nucleic acid molecule of the present invention into a plant (i.e., transformation) to achieve insect pest-inhibitory levels of expression of one or more iRNA molecules. A recombinant DNA molecule may, for example, be a vector, such as a linear or a closed circular plasmid. The vector system may be a single vector or plasmid, or two or more vectors or plasmids that together contain the total DNA to be introduced into the genome of a host. In addition, a vector may be an expression vector. Polynucleotides of the invention can, for example, be suitably inserted into a vector under the control of a suitable promoter that functions in one or more hosts to drive expression of a linked coding polynucleotide or other DNA element. Many vectors are available for this purpose, and selection of the appropriate vector will depend mainly on the size of the polynucleotide to be inserted into the vector and the particular host cell to be transformed with the vector. Each vector contains various components depending on its function (e.g., amplification of DNA or expression of DNA) and the particular host cell with which it is compatible.

To impart protection from an insect pest to a transgenic plant, a recombinant DNA may, for example, be transcribed into an iRNA molecule (e.g., a RNA molecule that forms a dsRNA molecule) within the tissues or fluids of the recombinant plant. An iRNA molecule may comprise a polyribonucleotide that is substantially identical and specifically hybridizable to a corresponding transcribed polyribonucleotide within an insect pest that may cause damage to the host plant species; for example, pollen beetle. The pest may contact the iRNA molecule that is transcribed in cells of the transgenic host plant, for example, by ingesting cells or fluids of the transgenic host plant that comprise the iRNA molecule. Thus, in particular examples, expression of a target gene is suppressed by the iRNA molecule within insect pests that infest the transgenic host plant. In some embodiments, suppression of expression of the target gene in an insect pest may result in the plant being protected from attack by the pest.

In order to enable delivery of iRNA molecules to an insect pest in a nutritional relationship with a plant cell that comprises a recombinant polynucleotide of the invention, expression (i.e., transcription) of iRNA molecules in the plant cell is typically required, although delivery may also be achieved, for example, by treating or coating the cell with a formulation comprising the iRNA molecules. Thus, a recombinant nucleic acid molecule may comprise a polynucleotide of the invention operably linked to one or more regulatory elements, such as a heterologous promoter element that functions in a host cell, such as a bacterial cell wherein the nucleic acid molecule is to be amplified or expressed, or a plant cell wherein the nucleic acid molecule is to be expressed.

Promoters suitable for use in nucleic acid molecules of the invention include those that are inducible, viral, synthetic, or constitutive, all of which are well known in the art. Non-limiting examples describing such promoters include U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-1 promoter); U.S. Pat. No. 6,232,526 (maize A3 promoter); U.S. Pat. No. 6,177,611 (constitutive maize promoters); U.S. Pat. Nos. 5,322,938, 5,352,605, 5,359,142, and 5,530,196 (CaMV 35S promoter); U.S. Pat. No. 6,433,252 (maize L3 oleosin promoter); U.S. Pat. No. 6,429,357 (rice actin 2 promoter, and rice actin 2 intron); U.S. Pat. No. 6,294,714 (light-inducible promoters); U.S. Pat. No. 6,140,078 (salt-inducible promoters); U.S. Pat. No. 6,252,138 (pathogen-inducible promoters); U.S. Pat. No. 6,175,060 (phosphorous deficiency-inducible promoters); U.S. Pat. No. 6,388,170 (bidirectional promoters); U.S. Pat. No. 6,635,806 (gamma-coixin promoter); and U.S. Patent Publication No. 2009/757,089 (maize chloroplast aldolase promoter). Additional promoters include the nopaline synthase (NOS) promoter (Ebert et al. (1987) Proc. Natl. Acad. Sci. USA 84(16):5745-9) and the octopine synthase (OCS) promoters (which are carried on tumor-inducing plasmids of Agrobacterium tumefaciens); the caulimovirus promoters such as the cauliflower mosaic virus (CaMV) 19S promoter (Lawton et al. (1987) Plant Mol. Biol. 9:315-24); the CaMV 35S promoter (Odell et al. (1985) Nature 313:810-2; the figwort mosaic virus 35S-promoter (Walker et al. (1987) Proc. Natl. Acad. Sci. USA 84(19):6624-8); the sucrose synthase promoter (Yang and Russell (1990) Proc. Natl. Acad. Sci. USA 87:4144-8); the R gene complex promoter (Chandler et al. (1989) Plant Cell 1:1175-83); the chlorophyll a/b binding protein gene promoter; CaMV 35S (U.S. Pat. Nos. 5,322,938, 5,352,605, 5,359,142, and 5,530,196); FMV 35S (U.S. Pat. Nos. 6,051,753, and 5,378,619); a PC1SV promoter (U.S. Pat. No. 5,850,019); the SCP1 promoter (U.S. Pat. No. 6,677,503); and AGRtu.nos promoters (GenBank™ Accession No. V00087; Depicker et al. (1982) J. Mol. Appl. Genet. 1:561-73; Bevan et al. (1983) Nature 304:184-7).

In particular embodiments, nucleic acid molecules of the invention comprise a tissue-specific promoter, such as a root-specific or leaf-specific promoter. In some embodiments, a polynucleotide for coleopteran pest control according to the invention may be cloned between two leaf-specific promoters oriented in opposite transcriptional directions relative to the polynucleotide or fragment, and which are operable in a transgenic plant cell and expressed therein to produce RNA molecules in the transgenic plant cell that subsequently may form dsRNA molecules, as described, supra. The iRNA molecules expressed in plant tissues may be ingested by an insect pest so that suppression of target gene expression is achieved.

Additional regulatory elements that may optionally be operably linked to a nucleic acid include 5′UTRs located between a promoter element and a coding polynucleotide that function as a translation leader element. The translation leader element is present in fully-processed mRNA, and it may affect processing of the primary transcript, and/or RNA stability. Examples of translation leader elements include maize and petunia heat shock protein leaders (U.S. Pat. No. 5,362,865), plant virus coat protein leaders, plant rubisco leaders, and others. See, e.g., Turner and Foster (1995) Molecular Biotech. 3(3):225-36. Non-limiting examples of 5′UTRs include GmHsp (U.S. Pat. No. 5,659,122); PhDnaK (U.S. Pat. No. 5,362,865); AtAntl; TEV (Carrington and Freed (1990) J. Virol. 64:1590-7); and AGRtunos (GenBank™ Accession No. V00087; and Bevan et al. (1983) Nature 304:184-7).

Additional regulatory elements that may optionally be operably linked to a nucleic acid also include 3′ non-translated elements, 3′ transcription termination regions, or polyadenylation regions. These are genetic elements located downstream of a polynucleotide, and include polynucleotides that provide polyadenylation signal, and/or other regulatory signals capable of affecting transcription or mRNA processing. The polyadenylation signal functions in plants to cause the addition of polyadenylate nucleotides to the 3′ end of the mRNA precursor. The polyadenylation element can be derived from a variety of plant genes, or from T-DNA genes. A non-limiting example of a 3′ transcription termination region is the nopaline synthase 3′ region (nos 3; Fraley et al. (1983) Proc. Natl. Acad. Sci. USA 80:4803-7). An example of the use of different 3′ non-translated regions is provided in Ingelbrecht et al., (1989) Plant Cell 1:671-80. Non-limiting examples of polyadenylation signals include one from a Pisum sativum RbcS2 gene (Ps.RbcS2-E9; Coruzzi et al. (1984) EMBO J. 3:1671-9) and AGRtu.nos (GenBank™ Accession No. E01312).

Some embodiments may include a plant transformation vector that comprises at least one of the above-described regulatory elements operatively linked to one or more polynucleotides of the present invention. When expressed, the one or more polynucleotides result in one or more iRNA molecule(s) comprising a polyribonucleotide that is specifically complementary or reverse complementary to all or part of a native RNA molecule in an insect pest. Thus, the polynucleotide(s) may comprise a segment encoding all or part of a polyribonucleotide present within a targeted insect pest RNA transcript, and may comprise inverted repeats of all or a part of a targeted transcript. A plant transformation vector may contain nucleotide sequences encoding polyribonucleotides that are specifically complementary to more than one target polynucleotide, thus allowing production of more than one dsRNA for inhibiting expression of two or more genes in cells of one or more populations or species of target insect pests. Polynucleotides comprising nucleotide sequences that encode polyribonucleotides that are specifically complementary or reverse complementary to fragments of different target genes can be combined into a single composite nucleic acid molecule for expression in a transgenic plant. Such segments may be contiguous or separated by a spacer.

In some embodiments, a plasmid already containing at least one polynucleotide(s) of the invention can be modified by the sequential insertion of additional polynucleotide(s) in the same plasmid, wherein the additional polynucleotide(s) are operably linked to the same regulatory elements as the original polynucleotide(s). In some embodiments, a construct may be designed for the inhibition of multiple target genes. In particular embodiments, the multiple genes to be inhibited are obtained from the same insect pest species (e.g., PB), which may enhance the effectiveness of the construct. In other embodiments, the genes can be derived from different insect pests, which may broaden the range of pests against which the construct is effective. When multiple genes are targeted for suppression or a combination of expression and suppression, a polycistronic DNA element can be engineered.

A recombinant nucleic acid molecule or vector of the present invention may comprise a selectable marker that confers a selectable phenotype on a transformed cell, such as a plant cell. Selectable markers may also be used to select for plants or plant cells that comprise a recombinant nucleic acid molecule of the invention. The marker may encode biocide resistance, antibiotic resistance (e.g., kanamycin, Geneticin (G418), bleomycin, hygromycin, etc.), or herbicide tolerance (e.g., glyphosate, etc.). Examples of selectable markers include, but are not limited to: a neo gene which codes for kanamycin resistance and can be selected for using kanamycin, G418, etc.; a bar gene which codes for bialaphos resistance; a mutant EPSP synthase gene which encodes glyphosate tolerance; a nitrilase gene which confers resistance to bromoxynil; a mutant acetolactate synthase (ALS) gene which confers imidazolinone or sulfonylurea tolerance; and a methotrexate resistant DHFR gene. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, streptomycin and tetracycline, and the like. Examples of such selectable markers are illustrated in, e.g., U.S. Pat. Nos. 5,550,318; 5,633,435; 5,780,708 and 6,118,047.

A recombinant nucleic acid molecule or vector of the present invention may also include a screenable marker. Screenable markers may be used to monitor expression. Exemplary screenable markers include a β-glucuronidase or uidA gene (GUS) which encodes an enzyme for which various chromogenic substrates are known (Jefferson et al. (1987) Plant Mol. Biol. Rep. 5:387-405); an R-locus gene, which encodes a product that regulates the production of anthocyanin pigments (red color) in plant tissues (Dellaporta et al. (1988) “Molecular cloning of the maize R-nj allele by transposon tagging with Ac.” In 18^{th} Stadler Genetics Symposium, P. Gustafson and R. Appels, eds. (New York: Plenum), pp. 263-82); a β-lactamase gene (Sutcliffe et al. (1978) Proc. Natl. Acad. Sci. USA 75:3737-41); a gene which encodes an enzyme for which various chromogenic substrates are known (e.g., PADAC, a chromogenic cephalosporin); a luciferase gene (Ow et al. (1986) Science 234:856-9); an xy/E gene that encodes a catechol dioxygenase that can convert chromogenic catechols (Zukowski et al. (1983) Gene 46(2-3):247-55); an amylase gene (Ikatu et al. (1990) Bio/Technol. 8:241-2); a tyrosinase gene which encodes an enzyme capable of oxidizing tyrosine to DOPA and dopaquinone which in turn condenses to melanin (Katz et al. (1983) J. Gen. Microbiol. 129:2703-14); and an α-galactosidase.

In some embodiments, recombinant nucleic acid molecules, as described, supra, may be used in methods for the creation of transgenic plants and expression of heterologous nucleic acids in plants to prepare transgenic plants that exhibit reduced susceptibility to insect pests. Plant transformation vectors can be prepared, for example, by inserting polynucleotides encoding iRNA molecules into plant transformation vectors and introducing these into plants.

Suitable methods for transformation of host cells include any method by which DNA can be introduced into a cell, such as by transformation of protoplasts (See, e.g., U.S. Pat. No. 5,508,184), by desiccation/inhibition-mediated DNA uptake (See, e.g., Potrykus et al. (1985) Mol. Gen. Genet. 199:183-8), by electroporation (See, e.g., U.S. Pat. No. 5,384,253), by agitation with silicon carbide fibers (See, e.g., U.S. Pat. Nos. 5,302,523 and 5,464,765), by Agrobacterium-mediated transformation (See, e.g., U.S. Pat. Nos. 5,563,055; 5,591,616; 5,693,512; 5,824,877; 5,981,840; and 6,384,301) and by acceleration of DNA-coated particles (See, e.g., U.S. Pat. Nos. 5,015,580; 5,550,318; 5,538,880; 6,160,208; 6,399,861; and 6,403,865), etc. Through the application of techniques such as these, the cells of virtually any species may be stably transformed. In some embodiments, transformation results in integration of a heterologous polynucleotide into the genome of the host cell. In the case of multicellular species, transgenic cells may be regenerated into a transgenic organism. Any of these techniques may be used to produce a transgenic plant, for example, comprising one or more polynucleotides encoding iRNA molecules in the genome of the transgenic plant.

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. The Ti (tumor-inducing)-plasmids contain a large segment, known as T-DNA, which is transferred to transformed plants. Another segment of the Ti plasmid, the Vir region, is responsible for T-DNA transfer. The T-DNA region is bordered by terminal repeats. In modified binary vectors, the tumor-inducing genes have been deleted, and the functions of the Vir region are utilized to transfer foreign DNA bordered by the T-DNA border elements. The T-region may also contain a selectable marker for efficient recovery of transgenic cells and plants, and a multiple cloning site for inserting polynucleotides for transfer such as a dsRNA encoding nucleic acid.

Thus, in some embodiments, a plant transformation vector is derived from a Ti plasmid of A. tumefaciens (See, e.g., U.S. Pat. Nos. 4,536,475, 4,693,977, 4,886,937, and 5,501,967; and European Patent No. EP 0 122 791) or a Ri plasmid of A. rhizogenes. Additional plant transformation vectors include, for example and without limitation, those described by Herrera-Estrella et al. (1983) Nature 303:209-13; Bevan et al. (1983) Nature 304:184-7; Klee et al. (1985) Bio/Technol. 3:637-42; and in European Patent No. EP 0 120 516, and those derived from any of the foregoing. Other bacteria such as Sinorhizobium, Rhizobium, and Mesorhizobium that interact with plants naturally can be modified to mediate gene transfer to a number of diverse plants. These plant-associated symbiotic bacteria can be made competent for gene transfer by acquisition of both a disarmed Ti plasmid and a suitable binary vector.

After tranforming recipient cells with a heterologous polynucleotide, transformed cells are generally identified for further culturing and plant regeneration. In order to improve the ability to identify transformed cells, one may desire to employ a selectable or screenable marker gene, as previously set forth, with the transformation vector used to generate the transformant. In the case where a selectable marker is used, transformed cells are identified within the potentially transformed cell population by exposing the cells to a selective agent or agents. In the case where a screenable marker is used, cells may be screened for the desired marker gene trait.

Cells that survive the exposure to the selective agent, or cells that have been scored positive in a screening assay, may be cultured in media that supports regeneration of plants. In some embodiments, any suitable plant tissue culture media (e.g., MS and N6 media) may be modified by including further substances, such as growth regulators. Tissue may be maintained on a basic medium with growth regulators until sufficient tissue is available to begin plant regeneration efforts, or following repeated rounds of manual selection, until the morphology of the tissue is suitable for regeneration (e.g., at least 2 weeks), then transferred to media conducive to shoot formation. Cultures are transferred periodically until sufficient shoot formation has occurred. Once shoots are formed, they are transferred to media conducive to root formation. Once sufficient roots are formed, plants can be transferred to soil for further growth and maturation.

To confirm the presence of a polynucleotide of interest (for example, a polynucleotide encoding one or more iRNA molecules that inhibit target gene expression in an insect pest) in the regenerating plants, a variety of assays may be performed. Such assays include, for example: molecular biological assays, such as Southern and northern blotting, PCR, and nucleic acid sequencing; biochemical assays, such as detecting the presence of a protein product, e.g., by immunological means (ELISA and/or western blots) or by enzymatic function; plant part assays, such as leaf or root assays; and analysis of the phenotype of the whole regenerated plant.

Integration events may be analyzed, for example, by PCR amplification using, e.g., oligonucleotide primers specific for a polynucleotide of interest. PCR genotyping is understood to include, but not be limited to, polymerase-chain reaction (PCR) amplification of gDNA derived from isolated host plant callus tissue predicted to contain a polynucleotide of interest integrated into the genome, followed by standard cloning and sequence analysis of PCR amplification products. Methods of PCR genotyping have been well described (for example, Rios, G. et al. (2002) Plant J. 32:243-53) and may be applied to gDNA derived from any plant species (e.g., B. napus) or tissue type, including cell cultures.

A transgenic plant formed using Agrobacterium-dependent transformation methods typically contains a single recombinant DNA inserted into one chromosome. The polynucleotide of the single recombinant DNA is referred to as a “transgenic event” or “integration event”. Such transgenic plants are heterozygous for the inserted heterologous polynucleotide. In some embodiments, a transgenic plant homozygous with respect to a transgene may be obtained by sexually mating (selfing) an independent segregant transgenic plant that contains a single exogenous gene to itself, for example a T₀ plant, to produce T₁ seed. One fourth of the T₁ seed produced will be homozygous with respect to the transgene. Germinating T₁ seed results in plants that can be tested for heterozygosity, typically using an SNP assay or a thermal amplification assay that allows for the distinction between heterozygotes and homozygotes (i.e., a zygosity assay).

In particular embodiments, at least 2, 3, 4, 5, 6, 7, 8, 9 or 10 or more different iRNA molecules are produced in a plant cell that have an insect pest-inhibitory effect. The iRNA molecules (e.g., dsRNA molecules) may be expressed from multiple polynucleotides introduced in different transformation events, or from a single polynucleotide introduced in a single transformation event. In some embodiments, a plurality of iRNA molecules are expressed under the control of a single promoter. In other embodiments, a plurality of iRNA molecules are expressed under the control of multiple promoters. Single iRNA molecules may be expressed that comprise multiple polyribonucleotides that are each at least substantially complementary or reverse complementary to different loci (for example, the locus defined by SEQ ID NOs:2-3) within one or more insect pests, both in different populations of the same species of insect pest, or in different species of insect pests.

In addition to direct transformation of a plant with a recombinant nucleic acid molecule, transgenic plants can be prepared by crossing a first plant having at least one transgenic event with a second plant lacking such an event. For example, a recombinant nucleic acid molecule comprising a polynucleotide that encodes an iRNA molecule may be introduced into a first plant line that is amenable to transformation to produce a transgenic plant comprising the polynucleotide, which transgenic plant may be crossed with a second plant line to introgress the polynucleotide that encodes the iRNA molecule into the second plant line.

In some aspects, seeds and commodity products produced by transgenic plants derived from transgenic plant cells are included, wherein the seeds or commodity products comprise a detectable amount of a polynucleotide or polyribonucleotide of the invention. In some embodiments, such commodity products may be produced, for example, by obtaining transgenic plants and preparing food or feed from them. Commodity products comprising one or more of the polynucleotides or polyribonucleotides of the invention include, for example and without limitation: meals, oils, crushed or whole grains or seeds of a plant, and any food product comprising any meal, oil, or crushed or whole grain of a transgenic plant or seed comprising one or more of the polynucleotides or polyribonucleotides of the invention. The detection of one or more of the polynucleotides or polyribonucleotides of the invention in one or more commodity or commodity products is de facto evidence that the commodity or commodity product is produced from a transgenic plant designed to express one or more of the iRNA molecules of the invention for the purpose of controlling insect pests.

In some embodiments, a transgenic plant or seed comprising a polynucleotide of the invention also may comprise at least one other transgenic event in its genome, including without limitation: a transgenic event from which is transcribed an iRNA molecule targeting a locus in a coleopteran pest other than the one defined by SEQ ID NOs:2-3, such as, for example, one or more loci selected from the group consisting of Caf1-180 (U.S. Patent Application Publication No. 2012/0174258), VatpaseC (U.S. Patent Application Publication No. 2012/0174259), Rho1 (U.S. Patent Application Publication No. 2012/0174260), VatpaseH (U.S. Patent Application Publication No. 2012/0198586), PPI-87B (U.S. Patent Application Publication No. 2013/0091600), RPA70 (U.S. Patent Application Publication No. 2013/0091601), RPS6 (U.S. Patent Application Publication No. 2013/0097730), ROP (U.S. patent application Publication Ser. No. 14/577,811), RNA polymerase I1 (U.S. Patent Application Publication No. 62/133,214), RNA polymerase II140 (U.S. patent application Publication Ser. No. 14/577,854), RNA polymerase II215 (U.S. Patent Application Publication No. 62/133,202), RNA polymerase II33 (U.S. Patent Application Publication No. 62/133,210), transcription elongation factor spt5 (U.S. Patent Application No. 62/168,613), transcription elongation factor spt6 (U.S. Patent Application No. 62/168,606), ncm (U.S. Patent Application No. 62/095,487), dre4 (U.S. patent application Ser. No. 14/705,807), COPI alpha (U.S. Patent Application No. 62/063,199), COPI beta (U.S. Patent Application No. 62/063,203), COPI gamma (U.S. Patent Application No. 62/063,192), and COPI delta (U.S. Patent Application No. 62/063,216); a transgenic event from which is transcribed an iRNA molecule targeting a gene in an organism other than a coleopteran pest (e.g., a plant-parasitic nematode); a gene encoding an insecticidal protein (e.g., a Bacillus thuringiensis insecticidal protein and a PIP-1 polypeptide); an herbicide tolerance gene (e.g., a gene providing tolerance to glyphosate); and a gene contributing to a desirable phenotype in the transgenic plant, such as increased yield, altered fatty acid metabolism, or restoration of cytoplasmic male sterility. In particular embodiments, polynucleotides encoding iRNA molecules of the invention may be combined with other insect control and disease traits in a plant to achieve desired traits for enhanced control of plant disease and insect damage. In some examples, combining insect control traits that employ distinct modes of action provides protected transgenic plants with superior and synergistic durability over plants harboring a single control trait, for example, because of the reduced probability that resistance to the trait(s) will develop in the field.

### V. Target Gene Suppression in an Insect Pest

A. Overview

In some embodiments of the invention, at least one nucleic acid molecule useful for the control of insect pests (e.g., pollen beetle) may be provided to an insect pest, wherein the nucleic acid molecule leads to RNAi-mediated gene silencing in the pest. In particular embodiments, an iRNA molecule (e.g., dsRNA, siRNA, miRNA, shRNA, and hpRNA) is provided to the pest. In some embodiments, a nucleic acid molecule useful for the control of insect pests may be provided to a pest by contacting the nucleic acid molecule with the pest. In specific embodiments, a nucleic acid molecule useful for the control of insect pests may be provided in a feeding substrate of the pest, for example, a nutritional composition. In specific embodiments, a nucleic acid molecule useful for the control of an insect pest may be provided through ingestion of plant material comprising the nucleic acid molecule that is ingested by the pest. In certain embodiments, the nucleic acid molecule is present in plant material through expression of a heterologous polynucleotide introduced into the plant material, for example, by transformation of a plant cell with a vector comprising the heterologous polynucleotide and regeneration of a plant material or whole plant from the transformed plant cell.

B. RNAi-Mediated Target Gene Suppression

In embodiments, the invention provides iRNA molecules (e.g., dsRNA, siRNA, miRNA, shRNA, and hpRNA) that may be designed to target essential native polynucleotides (e.g., prp19 mRNA) in the transcriptome of an insect pest (e.g., pollen beetle), for example by designing an iRNA molecule that comprises at least one strand comprising a polyribonucleotide that is specifically complementary or reverse complementary to the target polynucleotide. The sequence of an iRNA molecule so designed may be identical to that of the target polynucleotide, or may incorporate mismatches that do not prevent specific hybridization between the iRNA molecule and its target polynucleotide.

iRNA molecules of the invention may be used in methods for gene suppression in an insect pest, thereby reducing the level or incidence of damage caused by the pest on a plant (for example, a protected transgenic plant comprising an iRNA molecule). As used herein, the term “gene suppression” refers to any of the well-known methods for reducing the levels of protein produced as a result of gene transcription to mRNA and subsequent translation of the mRNA, including the reduction of protein expression from a gene or a coding polynucleotide including post-transcriptional inhibition of expression and transcriptional suppression. Post-transcriptional inhibition is mediated by specific homology between all or a part of an mRNA transcribed from a gene targeted for suppression and the corresponding iRNA molecule used for suppression. Additionally, post-transcriptional inhibition refers to the substantial and measurable reduction of the amount of mRNA available in the cell for binding by ribosomes.

In embodiments wherein the iRNA molecule of the invention is a dsRNA molecule, the dsRNA molecule may be cleaved by the enzyme, DICER, into short miRNA or siRNA molecules of approximately 20 nucleotides in length (e.g., from 19-23 nucleotides in length), such as those represented by SEQ ID NOs:17-31. A double-stranded siRNA molecule generated by DICER activity upon the dsRNA molecule may be separated into two single-stranded siRNAs, the “passenger strand” and the “guide strand.” The passenger strand may be degraded, and the guide strand may be incorporated into RISC. Post-transcriptional inhibition occurs by specific hybridization of the guide strand with an mRNA molecule, and subsequent cleavage by the enzyme, Argonaute (catalytic component of the RISC complex).

In embodiments of the invention, any form of iRNA molecule may be used. Those of skill in the art will understand that dsRNA molecules typically are more stable during preparation and during the step of providing the iRNA molecule to a cell than are single-stranded RNA molecules, and are typically also more stable in a cell. Thus, while siRNA and miRNA molecules, for example, may be equally effective in some embodiments, a dsRNA molecule may be chosen due to its stability. Certain embodiments include polynucleotides that encode only one strand of a dsRNA molecules, for example, such that they may be combined in a transgenic cell with a polynucleotide encoding the other strand of the dsRNA molecule, wherein the dsRNA molecule is formed in the cell by hybridization of the two strands encoded by the separate polynucleotides.

In particular embodiments, a nucleic acid molecule is provided that comprises a polynucleotide, which polynucleotide may be expressed in vitro to produce an iRNA molecule that comprises a polyribonucleotide that is substantially homologous to a polyribonucleotide of an RNA molecule encoded by a polynucleotide within the genome of an insect pest. In certain embodiments, the in vitro transcribed iRNA molecule may be a stabilized dsRNA molecule that comprises a stem-loop structure. After an insect pest contacts the in vitro transcribed iRNA molecule, post-transcriptional inhibition of a target gene in the pest may occur.

In some embodiments of the invention, expression of a polynucleotide comprising at least 15 contiguous nucleotides (e.g., at least 19 contiguous nucleotides) of a target gene or its complement or reverse complement are used in a method for post-transcriptional inhibition of the target gene in an insect pest, wherein the polynucleotide is selected from the group consisting of: SEQ ID NO:1; the complement or reverse complement of SEQ ID NO:1; the PB prp19 coding sequence comprising SEQ ID NOs:2-3; the complement or reverse complement of the PB prp19 coding sequence comprising SEQ ID NOs:2-3; a fragment of at least 15 contiguous nucleotides of the PB prp19 coding sequence comprising SEQ ID NOs:2-3 (e.g., SEQ ID NO:4); the complement of a fragment of at least 15 contiguous nucleotides of the PB prp19 coding sequence comprising SEQ ID

NOs:2-3; the reverse complement of a fragment of at least 15 contiguous nucleotides of the PBprp19 coding sequence comprising SEQ ID NOs:2-3; a native coding polynucleotide of a Meligethes organism (e.g., PB) comprising SEQ ID NO:4; the complement of a native coding polynucleotide of a Meligethes organism comprising SEQ ID NO:4; the reverse complement of a native coding polynucleotide of a Meligethes organism comprising SEQ ID NO:4; a fragment of at least 15 contiguous nucleotides of a native coding polynucleotide of a Meligethes organism comprising SEQ ID NO:4; the complement of a fragment of at least 15 contiguous nucleotides of a native coding polynucleotide of a Meligethes organism comprising SEQ ID NO:4; and the reverse complement of a fragment of at least 15 contiguous nucleotides of a native coding polynucleotide of a Meligethes organism comprising SEQ ID NO:4. In certain embodiments, expression of a nucleic acid molecule that is at least about 80% identical (e.g., 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, and 100%) with any of the foregoing may be used. In these and further embodiments, a nucleic acid molecule may be expressed that specifically hybridizes to an RNA molecule present in at least one cell of an insect pest.

It is an important feature of some embodiments herein that the RNAi post-transcriptional inhibition system is able to tolerate sequence variations among target genes that might be expected due to genetic mutation, strain polymorphism, or evolutionary divergence. An iRNA molecule may not need to be absolutely identical to either a primary transcription product or a fully-processed mRNA of a target gene (or the complements and reverse complements thereof), so long as the iRNA molecule is specifically hybridizable to either a primary transcription product or a fully-processed mRNA of the target gene. Moreover, the iRNA molecule need not be full-length, relative to either a primary transcription product or a fully processed mRNA of the target gene.

Inhibition of a target gene using the iRNA technology of the present invention is sequence-specific; i.e., polynucleotides substantially identical to the iRNA molecule(s) or their complements or reverse complements are targeted for genetic inhibition. In some embodiments, an RNA molecule comprising a polyribonucleotide with a nucleotide sequence that is identical to that of a portion of an mRNA transcribed from a target gene, or its complement or reverse complement, may be used for inhibition. In these and further embodiments, an RNA molecule comprising a polyribonucleotide with one or more insertion, deletion, and/or point mutations relative to a target polynucleotide may be used. In particular embodiments, an iRNA molecule and a portion of a target gene, or its complement or reverse complement, may share, for example, at least from about 80%, at least from about 81%, at least from about 82%, at least from about 83%, at least from about 84%, at least from about 85%, at least from about 86%, at least from about 87%, at least from about 88%, at least from about 89%, at least from about 90%, at least from about 91%, at least from about 92%, at least from about 93%, at least from about 94%, at least from about 95%, at least from about 96%, at least from about 97%, at least from about 98%, at least from about 99%, at least from about 100%, and 100% sequence identity. In some examples, the duplex region of a dsRNA molecule may be specifically hybridizable with a portion of a target gene transcript. In specifically hybridizable molecules, a less than full length polyribonucleotide exhibiting a greater degree of sequence identity compensates for a longer, less identical polyribonucleotide. The length of a polyribonucleotide of a duplex region of a dsRNA molecule that is identical or substantially identical to a portion of a target gene transcript, or the complement or reverse complement thereof, may be at least about 25, 50, 100, 200, 300, 400, 500, or at least about 1000 bases. In some examples, a polyribonucleotide of greater than 20-100 nucleotides may be used. In particular examples, a polyribonucleotide of greater than about 200-300 nucleotides may be used. In these and further particular examples, a polyribonucleotide of greater than about 500-1000 nucleotides may be used, depending on the size of the target gene.

In certain embodiments, expression of a target gene in an insect pest may be inhibited by at least 10%; at least 33%; at least 50%; or at least 80% within a cell of the pest, such that a significant inhibition takes place. Significant inhibition refers to inhibition over a threshold that results in a detectable phenotype (e.g., cessation of growth, cessation of feeding, cessation of development, induced mortality, etc.), or a detectable decrease in RNA and/or gene product corresponding to the target gene being inhibited. Although, in certain embodiments of the invention, inhibition occurs in substantially all cells of the pest, in other embodiments, inhibition occurs only in a subset of cells expressing the target gene.

In some embodiments, transcriptional suppression is mediated by the presence in a cell of a dsRNA molecule exhibiting substantial sequence identity to a promoter DNA or the complement thereof to effect what is referred to as “promoter trans suppression.” Gene suppression may be effective against target genes in an insect pest that may ingest or contact such dsRNA molecules, for example, by ingesting or contacting plant material containing the dsRNA molecules. dsRNA molecules for use in promoter trans suppression may be specifically designed to inhibit or suppress the expression of one or more homologous or complementary polynucleotides in the cells of the insect pest. Post-transcriptional gene suppression by antisense or sense oriented RNA to regulate gene expression in plant cells is disclosed in U.S. Pat. Nos. 5,107,065; 5,759,829; 5,283,184; and 5,231,020.

C. Expression of iRNA Molecules Provided to an Insect Pest

Expression of iRNA molecules for RNAi-mediated gene inhibition in an insect pest may be carried out in any one of many in vitro or in vivo formats. The iRNA molecules may then be provided to an insect pest, for example, by contacting the iRNA molecules with the pest, or by causing the pest to ingest or otherwise internalize the iRNA molecules. Some embodiments include transgenic host plants of the insect pest, transgenic plant cells of the plants, and progeny of transgenic plants. The transgenic plant cells and transgenic plants may be engineered to express one or more of the iRNA molecules, for example, under the control of a heterologous promoter, to provide a pest-protective effect. Thus, when a transgenic plant or plant cell is consumed by an insect pest during feeding, the pest may ingest iRNA molecules expressed in the transgenic plants or cells. The polynucleotides of the present invention may also be introduced into a wide variety of prokaryotic and eukaryotic microorganism hosts to produce iRNA molecules. The term “microorganism” includes prokaryotic and eukaryotic species, such as bacteria and fungi.

Modulation of gene expression may include partial or complete suppression of such expression. In some embodiments, a method for suppression of gene expression in an insect pest comprises providing in the tissue of a host of the pest a gene-suppressive amount of at least one dsRNA molecule formed following transcription of a polynucleotide as described herein, at least one segment of which is complementary to an mRNA within the cells of the insect pest. A dsRNA molecule, including its modified form such as an siRNA, miRNA, shRNA, or hpRNA molecule, ingested by an insect pest may be at least from about 80%, 81%, 82%, 83%, 84%, 85%, 86%, 87%, 88%, 89%, 90%, 91%, 92%, 93%, 94%, 95%, 96%, 97%, 98%, 99%, or about 100% identical to an RNA molecule transcribed from a PB prp19 gene, for example, comprising SEQ ID NOs:2-3. Isolated and substantially purified nucleic acid molecules including, but not limited to, non-naturally occurring polynucleotides and recombinant DNA constructs for providing dsRNA molecules are therefore provided, which suppress or inhibit the expression of a target endogenous coding polynucleotide in an insect pest when introduced thereto.

Particular embodiments provide a delivery system for the delivery of iRNA molecules for the post-transcriptional inhibition of one or more target gene(s) in an insect plant pest and control of a population of the plant pest. In some embodiments, the delivery system comprises ingestion of a host transgenic plant cell or contents of the host cell comprising RNA molecules transcribed in the host cell. In these and further embodiments, a transgenic plant cell or a transgenic plant is created that contains a recombinant DNA construct encoding a stabilized dsRNA molecule of the invention. Transgenic plant cells and transgenic plants comprising nucleic acids encoding a particular iRNA molecule may be produced by employing recombinant DNA technologies (which basic technologies are well-known in the art) to construct a plant transformation vector comprising a polynucleotide encoding an iRNA molecule of the invention (e.g., a stabilized dsRNA molecule); to transform a plant cell or plant; and to generate the transgenic plant cell or the transgenic plant that contains the transcribed iRNA molecule.

To impart protection from insect pests to a transgenic plant, a recombinant DNA molecule may, for example, be transcribed into an iRNA molecule, such as a dsRNA molecule, a siRNA molecule, a miRNA molecule, a shRNA molecule, or a hpRNA molecule. In some embodiments, a RNA molecule transcribed from a recombinant DNA may form a dsRNA molecule within the tissues or fluids of the recombinant plant. Such a dsRNA molecule may be comprised in part of a polyribonucleotide that is identical to a corresponding target polyribonucleotide transcribed from a DNA within an insect pest of a type that may infest the host plant. Expression of a target gene within the pest is suppressed by the dsRNA molecule, and the suppression of expression of the target gene in the pest results in the transgenic plant being resistant to the pest. The modulatory effects of dsRNA molecules have been shown to be applicable to a variety of genes expressed in pests, including, for example, endogenous genes responsible for cellular metabolism or cellular transformation, including house-keeping genes; transcription factors; molting-related genes; and other genes which encode polypeptides involved in cellular metabolism or normal growth and development.

For transcription from a transgene in vivo or an expression construct, a regulatory region (e.g., promoter, enhancer, silencer, and polyadenylation signal) may be used in some embodiments to transcribe the RNA strand (or strands). Therefore, in some embodiments, as set forth, supra, a polynucleotide for use in producing iRNA molecules may be operably linked to one or more promoter elements functional in a plant host cell. The promoter may be an endogenous promoter, normally resident in the host genome. The polynucleotide of the present invention, under the control of an operably linked promoter element, may further be flanked by additional elements that advantageously affect its transcription and/or the stability of a resulting transcript. Such elements may be located upstream of the operably linked promoter, downstream of the 3′ end of the expression construct, and may occur both upstream of the promoter and downstream of the 3′ end of the expression construct.

Some embodiments provide methods for reducing the damage to a host plant (e.g., a canola plant) caused by an insect pest that feeds on the plant, wherein the method comprises providing in the host plant a transgenic plant cell expressing at least one nucleic acid molecule of the invention, wherein the nucleic acid molecule functions upon being taken up by the pest(s) to inhibit the expression of a target polynucleotide within the pest(s), which inhibition of expression results in mortality and/or reduced growth of the pest(s), thereby reducing the damage to the host plant caused by the pest(s). In some embodiments, the nucleic acid molecule is a dsRNA molecule. In particular embodiments, the dsRNA molecule comprises more than one polyribonucleotide that is specifically hybridizable to a nucleic acid molecule expressed in an insect pest cell. In some embodiments, the nucleic acid molecule comprises one polyribonucleotide that is specifically hybridizable to a nucleic acid molecule expressed in an insect pest cell.

In some embodiments, a method for increasing the yield of a crop plant (e.g., a Brassica plant, such as canola) is provided, wherein the method comprises introducing into the crop plant at least one nucleic acid molecule comprising a polynucleotide of the invention; and cultivating the crop plant to allow the expression of an iRNA molecule from the polynucleotide, wherein expression of an iRNA molecule inhibits insect pest damage and/or growth, thereby reducing or eliminating a loss of yield due to pest infestation. In some embodiments, the iRNA molecule is a dsRNA molecule. In these and further embodiments, the dsRNA molecules may each comprise more than one polyribonucleotide that is specifically hybridizable to a nucleic acid molecule expressed in an insect pest cell. Thus, specific polyribonucleotides of a dsRNA molecule may be expressed from one or more nucleotide sequences within a polynucleotide of the invention.

In some embodiments, a method for modulating the expression of a target gene in an insect pest is provided, the method comprising: transforming a plant cell with a vector comprising a polynucleotide encoding at least one iRNA molecule of the invention, wherein the polynucleotide is operatively-linked to a promoter and a transcription termination element; culturing the transformed plant cell under conditions sufficient to allow for development of a plant cell culture including a plurality of transgenic plant cells; selecting for transgenic plant cells that have integrated the polynucleotide into their genomes; screening the transgenic plant cells for expression of the iRNA molecule encoded by the integrated polynucleotide; selecting a transgenic plant cell that expresses the iRNA molecule; and feeding the selected transgenic plant cell to the insect pest. Plants may also be regenerated from transgenic plant cells that express an iRNA molecule encoded by the integrated polynucleotide. In some embodiments, the iRNA molecule is a dsRNA molecule comprising a polyribonucleotide that is specifically hybridizable to the transcript of a target gene in the insect pest. In these and further embodiments, the dsRNA molecule comprises more than one polyribonucleotide that is transcribed from a nucleotide sequence within the polynucleotide encoding the dsRNA molecule.

iRNA molecules of the invention can be incorporated within the seeds of a plant species (e.g., a Brassica sp.), either as a product of expression from a heterologous polynucleotide incorporated into a genome of the plant cells, or as incorporated into a coating or seed treatment that is applied to the seed before planting. A plant cell comprising a heterologous polynucleotide of the invention is considered to comprise a transgenic event. Also included in embodiments of the invention are delivery systems for the delivery of iRNA molecules to insect pests. For example, the iRNA molecules of the invention may be directly introduced into the cells of a pest(s). Methods for introduction may include direct mixing of iRNA with plant tissue from a host for the insect pest(s), as well as application of compositions comprising iRNA molecules of the invention to host plant tissue. For example, iRNA molecules may be sprayed onto a plant surface. Alternatively, an iRNA molecule may be expressed by a microorganism, and the microorganism may be applied onto the plant surface, or introduced into a root or stem by a physical means such as an injection. As discussed, supra, a transgenic plant may also be genetically engineered to express at least one iRNA molecule in an amount sufficient to kill insect pests infesting the plant. iRNA molecules produced by chemical or enzymatic synthesis may also be formulated in a manner consistent with common agricultural practices, and used as spray-on or bait products for controlling plant damage by an insect pest. The formulations may include the appropriate adjuvants (e.g., stickers and wetters) required for efficient foliar coverage, as well as UV protectants to protect iRNA molecules from UV damage. Such additives are commonly used in the bioinsecticide industry, and are well-known to those skilled in the art. Such applications may be combined with other spray-on insecticide applications (biologically based or otherwise) to enhance plant protection from the pests.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

The following EXAMPLES are provided to illustrate certain particular features and/or aspects. These EXAMPLES should not be construed to limit the disclosure to the particular features or aspects described.

### EXAMPLES

### Example 1: Pollen Beetle Transcriptome

Insects.

Larvae and adult pollen beetles were collected from fields with flowering rapeseed plants (Giessen, Germany). Young adult beetles (each per treatment group: n=20; 3 replicates) were challenged by injecting a mixture of two different bacteria (Staphylococcus aureus and Pseudomonas aeruginosa), one yeast (Saccharomyces cerevisiae) and bacterial LPS. Bacterial cultures were grown at 37° C. with agitation, and the optical density was monitored at 600 nm (OD600). The cells were harvested at OD600 ˜1 by centrifugation and resuspended in phosphate-buffered saline. The mixture was introduced ventrolaterally by pricking the abdomen of pollen beetle imagoes using a dissecting needle dipped in an aqueous solution of 10 mg/ml LPS (purified E. coli endotoxin; SIGMA, Taufkirchen, Germany) and the bacterial and yeast cultures. Along with the immune challenged beetles, naïve beetles, and larvae were collected (n=20 per and 3 replicates each) at the same time point.

RNA Isolation.

Total RNA was extracted 8 h after immunization from frozen beetles and larvae using TriReagent (Molecular Research Centre, Cincinnati, Ohio, USA) and purified using the RNeasy Micro Kit (QIAGEN, Hilden, Germany) in each case following the manufacturers' guidelines. The integrity of the RNA was verified using an Agilent 2100 Bioanalyzer and a RNA 6000 Nano Kit (AGILENT TECHNOLOGIES, Palo Alto, Calif., USA). The quantity of RNA was determined using a Nanodrop ND-1000 spectrophotometer. RNA was extracted from each of the adult immune-induced treatment groups, adult control groups, and larval groups individually and equal amounts of total RNA were subsequently combined in one pool per sample (immune-challenged adults, control adults and larvae) for sequencing.

Transcriptome Information.

RNA-Seq data generation and assembly Single-read 100-bp RNA-Seq was carried out separately on 5 μg total RNA isolated from immune-challenged adult beetles, naïve (control) adult beetles, and untreated larvae. Sequencing was carried out by EUROFINS MWG Operon using the Illumina HiSeq-2000 platform. This yielded 20.8 million reads for the adult control beetle sample, 21.5 million reads for the LPS-challenged adult beetle sample and 25.1 million reads for the larval sample. The pooled reads (67.5 million) were assembled using Velvet/Oases assembler software (Schulz et al. (2012) Bioinformatics. 28:1086-92; Zerbino and Birney (2008) Genome Res. 18:821-9). The transcriptome contained 55,648 sequences.

### Example 2: Mortality of Pollen Beetle Following Treatment with Prp19 iRNA

Gene-specific primers including the T7 polymerase promoter sequence at the 5′ end were used to create PCR products of approximately 306 bp by PCR (SEQ ID NO:4). PCR fragments were cloned in the pGEM T easy vector according to the manufacturer's protocol and sent to a sequencing company to verify the sequence. The dsRNA was then produced by the T7 RNA polymerase (MEGAscript® RNAi Kit, Applied Biosystems) from a PCR construct generated from the sequenced plasmid according to the manufacturer's protocol.

Injection Bioassay.

Injection of −100 nL dsRNA (1 μg/uL) into adult beetles (Table 1) was performed with a micromanipulator under a dissecting stereomicroscope. Animals were anaesthetized on ice before they were affixed to double-stick tape. Controls received the same volume of water. All controls in all stages could not be tested due to a lack of animals. Controls were performed on a different date due to the limited availability of insects. Pollen beetles were maintained in Petri dishes with dried pollen and a wet tissue.

TABLE 1
Results of M. aeneus adult pollen beetle injection bioassay. (Percentage of
survival mean ± standard deviation (SD), n = 3 groups of 10)

| | % Survival Mean ± SD | | | | |
|---|---|---|---|---|---|
| Treatment | Day 0 | Day 2 | Day 4 | Day 6 | Day 8 |
| prp19 | 100 ± 0 | 97 ± 6 | 87 ± 15 | 77 ± 15 | 50 ± 10 |
| Control | 100 ± 0 | 100 ± 0 | 100 ± 0 | 97 ± 6 | 87 ± 6 |
| | Day 10 | Day 12 | Day 14 | Day 16 | |
| prp19 | 13 ± 12 | 7 ± 12 | 3 ± 6 | 3 ± 6 | |
| Control | 77 ± 15 | 77 ± 12 | 77 ± 12 | 77 ± 12 | |

Feeding Bioassay.

Beetles were kept without access to water in empty falcon tubes 24 h before treatment, and then fed with prp19 dsRNA. Table 2. A droplet of dsRNA (˜5 μL) was placed in a small Petri dish, and 5 to 8 beetles were added to the Petri dish. Animals were observed under a stereomicroscope, and those that ingested dsRNA containing diet solution were selected for the bioassay. Beetles were transferred into petri dishes with dried pollen and a wet tissue. Controls received the same volume of water. All controls in all stages could not be tested due to a lack of animals. Controls were performed on a different date due to the limited availability of insects.

TABLE 2
Results of M. aeneus adult feeding bioassay. (Percentage of survival
mean ± standard deviation (SD), n = 3 groups of 10)

| | % Survival Mean ± SD | | | | |
|---|---|---|---|---|---|
| Treatment | Day 0 | Day 2 | Day 4 | Day 6 | Day 8 |
| prp19 | 100 ± 0 | 100 ± 0 | 100 ± 0 | 97 ± 6 | 83 ± 12 |
| Control | 100 ± 0 | 100 ± 0 | 100 ± 0 | 90 ± 10 | 87 ± 12 |
| | Day 10 | Day 12 | Day 14 | Day 16 | |
| prp19 | 77 ± 6 | 73 ± 6 | 70 ± 0 | 53 ± 21 | |
| Control | 87 ± 12 | 87 ± 12 | 87 ± 12 | 87 ± 12 | |

### Example 3: Agrobacterium-Mediated Transformation of Canola Hypocotyls

10-20 transgenic Brassica napus plants comprising an RNAi construct that encodes hairpin dsRNA targetingprp19 are generated for pollen beetle challenge. A hairpin dsRNA-encoding polynucleotide comprising a contiguous nucleotide sequence of PB prp19 (e.g., SEQ ID NO:4) is SEQ ID NO:11.

Agrobacterium Preparation.

The Agrobacterium strain containing the binary plasmid is streaked out on YEP media (Bacto Peptone™ 20.0 gm/L and Yeast Extract 10.0 gm/L) plates containing streptomycin (100 mg/mL) and spectinomycin (50 mg/mL) and incubated for 2 days at 28° C. The propagated Agrobacterium strain containing the binary plasmid is scraped from the 2-day streak plate using a sterile inoculation loop. The scraped Agrobacterium strain containing the binary plasmid is then inoculated into 150 mL modified YEP liquid with streptomycin (100 mg/mL) and spectinomycin (50 mg/mL) into sterile 500 mL baffled flask(s) and shaken at 200 rpm at 28° C. The cultures are centrifuged and resuspended in M-medium (LS salts, 3% glucose, modified B5 vitamins, 1 μM kinetin, 1 μM 2,4-D, pH 5.8) and diluted to the appropriate density (50 Klett Units as measured using a spectrophotometer) prior to transformation of canola hypocotyls.

Canola Transformation

Seed Germination:

Canola seeds (var. NEXERA 710™) are surface-sterilized in 10% Clorox™ for 10 minutes and rinsed three times with sterile distilled water (seeds are contained in steel strainers during this process). Seeds are planted for germination on ½ MS Canola medium (½ MS, 2% sucrose, 0.8% agar) contained in Phytatrays™ (25 seeds per Phytatray™) and placed in a Percival™ growth chamber with growth regime set at 25° C., photoperiod of 16:8 hours light:dark for 5 days of germination.

Pre-Treatment:

On day 5, hypocotyl segments of about 3 mm in length are aseptically excised, the remaining root and shoot sections are discarded (drying of hypocotyl segments is prevented by immersing the hypocotyls segments into 10 mL sterile milliQ™ water during the excision process). Hypocotyl segments are placed horizontally on sterile filter paper on callus induction medium, MSK1D1 (MS, 1 mg/L kinetin, 1 mg/L 2,4-D, 3.0% sucrose, 0.7% phytagar) for 3 days pre-treatment in a Percival™ growth chamber with growth regime set at 22-23° C., and a photoperiod of 16:8 hours light:dark.

Co-Cultivation with Agrobacterium:

The day before Agrobacterium co-cultivation, flasks of YEP medium containing the appropriate antibiotics, are inoculated with the Agrobacterium strain containing the binary plasmid. Hypocotyl segments are transferred from filter paper callus induction medium, MSK1D1 to an empty 100×25 mm Petri™ dishes containing 10 mL liquid M-medium to prevent the hypocotyl segments from drying. A spatula is used at this stage to scoop the segments and transfer the segments to new medium. The liquid M-medium is removed with a pipette and 40 mL Agrobacterium suspension is added to the Petri™ dish (500 segments with 40 mL Agrobacterium solution). The hypocotyl segments are treated for 30 minutes with periodic swirling of the Petri™ dish, so that the hypocotyl segments remained immersed in the Agrobacterium solution. At the end of the treatment period, the Agrobacterium solution is pipetted into a waste beaker; autoclaved and discarded (the Agrobacterium solution is completely removed to prevent Agrobacterium overgrowth). The treated hypocotyls are transferred with forceps back to the original plates containing MSK1D1 media overlaid with filter paper (care is taken to ensure that the segments did not dry). The transformed hypocotyl segments and non-transformed control hypocotyl segments are returned to the Percival™ growth chamber under reduced light intensity (by covering the plates with aluminum foil), and the treated hypocotyl segments are co-cultivated with Agrobacterium for 3 days.

Callus Induction on Selection Medium:

After 3 days of co-cultivation, the hypocotyl segments are individually transferred with forceps onto callus induction medium, MSK1D1H1 (MS, 1 mg/L kinetin, 1 mg/L 2,4-D, 0.5 gm/L MES, 5 mg/L AgNO3, 300 mg/L Timentin™, 200 mg/L carbenicillin, 1 mg/L Herbiace™, 3% sucrose, 0.7% phytagar) with growth regime set at 22-26° C. The hypocotyl segments are anchored on the medium, but are not deeply embedded into the medium.

Selection and Shoot Regeneration:

After 7 days on callus induction medium, the callusing hypocotyl segments are transferred to Shoot Regeneration Medium 1 with selection, MSB3Z1H1 (MS, 3 mg/L BAP, 1 mg/L zeatin, 0.5 gm/L MES, 5 mg/L AgNO3, 300 mg/L Timentin™, 200 mg/L carbenicillin, 1 mg/L Herbiace™, 3% sucrose, 0.7% phytagar). After 14 days, the hypocotyl segments which develop shoots are transferred to Regeneration Medium 2 with increased selection, MSB3Z1H3 (MS, 3 mg/L BAP, 1 mg/L Zeatin, 0.5 gm/L MES, 5 mg/L AgNO3, 300 mg/l Timentin™, 200 mg/L carbenicillin, 3 mg/L Herbiace™, 3% sucrose, 0.7% phytagar) with growth regime set at 22-26° C.

Shoot Elongation:

After 14 days, the hypocotyl segments that develop shoots are transferred from Regeneration Medium 2 to shoot elongation medium, MSMESH5 (MS, 300 mg/L Timentin™, 5 mg/L Herbiace™, 2% sucrose, 0.7% TC Agar) with growth regime set at 22-26° C. Shoots that are already elongated are isolated from the hypocotyl segments and transferred to MSMESH5. After 14 days, the remaining shoots which have not elongated in the first round of culturing on shoot elongation medium are transferred to fresh shoot elongation medium MSMESH5. At this stage all remaining hypocotyl segments which do not produce shoots are discarded.

Root Induction:

After 14 days of culturing on the shoot elongation medium, the isolated shoots are transferred to MSMEST medium (MS, 0.5 g/L MES, 300 mg/L Timentin™, 2% sucrose, 0.7% TC Agar) for root induction at 22-26° C. Any shoots which do not produce roots after incubation in the first transfer to MSMEST medium are transferred for a second or third round of incubation on MSMEST medium until the shoots develop roots.

### Example 4: Transgenic Plants Comprising Pollen Beetle Pest Control Polynucleotides

Transgenic plants are generated that express hairpin dsRNA targeting PB prp19. Hairpin dsRNA-encoding polynucleotides comprise a nucleotide sequence that is at least 15 (e.g., at least 19) nucleotides in length and are a contiguous fragment of the PB prp19 polynucleotide comprising SEQ ID NOs:2-3. Additional hairpin dsRNAs are derived, for example, from coleopteran pest sequences such as, for example, Caf1-180 (U.S. Patent Application Publication No. 2012/0174258), VatpaseC (U.S. Patent Application Publication No. 2012/0174259), Rho1 (U.S. Patent Application Publication No. 2012/0174260), VatpaseH (U.S. Patent Application Publication No. 2012/0198586), PPI-87B (U.S. Patent Application Publication No. 2013/0091600), RPA70 (U.S. Patent Application Publication No. 2013/0091601), RPS6 (U.S. Patent Application Publication No. 2013/0097730), ROP (U.S. patent application Publication Ser. No. 14/577,811), RNA polymerase I1 (U.S. Patent Application Publication No. 62/133,214), RNA polymerase II140 (U.S. patent application Publication Ser. No. 14/577,854), RNA polymerase II215 (U.S. Patent Application Publication No. 62/133,202), RNA polymerase II33 (U.S. Patent Application Publication No. 62/133,210), transcription elongation factor spt5 (U.S. Patent Application No. 62/168,613), transcription elongation factor spt6 (U.S. Patent Application No. 62/168,606), ncm (U.S. Patent Application No. 62/095,487), dre4 (U.S. patent application Ser. No. 14/705,807), COPI alpha (U.S. Patent Application No. 62/063,199), COPI beta (U.S. Patent Application No. 62/063,203), COPI gamma (U.S. Patent Application No. 62/063,192), and COPI delta (U.S. Patent Application No. 62/063,216). These are confirmed through RT-PCR or other molecular analysis methods.

Total RNA preparations from selected independent Ti lines are optionally used for RT-PCR with primers designed to bind in the linker of the hairpin expression cassette in each of the RNAi constructs. In addition, specific primers for each target gene in an RNAi construct are optionally used to amplify and confirm the production of the pre-processed mRNA required for siRNA production in planta. The amplification of the desired bands for each target gene confirms the expression of the hairpin RNA in each transgenic plant. Processing of the dsRNA hairpin of the target genes into siRNA is subsequently optionally confirmed in independent transgenic lines using RNA blot hybridizations.

Moreover, RNAi molecules having mismatch sequences with more than 80% sequence identity to target genes affect coleopteran insects in a way similar to that seen with RNAi molecules having 100% sequence identity to the target genes. The pairing of mismatch sequence with native sequences to form a hairpin dsRNA in the same RNAi construct delivers plant-processed siRNAs capable of affecting the growth, development, and viability of feeding coleopteran pests.

In planta delivery of dsRNA, siRNA, or miRNA corresponding to target genes and the subsequent uptake by coleopteran pests through feeding results in down-regulation of the target genes in the coleopteran pest through RNA-mediated gene silencing. When the function of a target gene is important at one or more stages of development, the growth and/or development of the coleopteran pest is affected, and in the case of Meligethes aeneus, leads to failure to successfully infest, feed, and/or develop, or leads to death of the coleopteran pest. The choice of target genes and the successful application of RNAi are then used to control coleopteran pests.

Phenotypic Comparison of Transgenic RNAi Lines and Non-Transformed Plants.

Target coleopteran pest genes or sequences selected for creating hairpin dsRNA have no similarity to any known plant gene sequence. Hence, it is not expected that the production or the activation of (systemic) RNAi by constructs targeting these coleopteran pest genes or sequences will have any deleterious effect on transgenic plants. However, development and morphological characteristics of transgenic lines are compared with non-transformed plants, as well as those of transgenic lines transformed with an “empty” vector having no hairpin-expressing gene. Plant root, shoot, foliage and reproduction characteristics are compared. There is no observable difference in root length and growth patterns of transgenic and non-transformed plants. Plant shoot characteristics such as height, leaf numbers and sizes, time of flowering, floral size and appearance are similar. In general, there are no observable morphological differences between transgenic lines and those without expression of target iRNA molecules when cultured in vitro and in soil in the glasshouse.

### Example 5: Transgenic Plants Comprising a Pollen Beetle

Pest Control Polynucleotide and Additional RNAi Constructs

A transgenic plant comprising a heterologous coding sequence in its genome that is transcribed into an iRNA molecule that targets an organism other than pollen beetle (for example, at least one dsRNA molecule targeting a gene other than the PB gene comprising SEQ ID NOs:2-3) is produced by secondary transformation via Agrobacterium or WHISKERS™ methodologies (see Petolino and Arnold (2009) Methods Mol. Biol. 526:59-67) to produce additional insecticidal dsRNA molecules. For this, plant transformation plasmid vectors are delivered via Agrobacterium or WHISKERS™-mediated transformation methods into suspension cells or immature embryos obtained from a transgenic plant comprising a heterologous coding sequence in its genome that is transcribed into an iRNA molecule that targets the PB gene comprising SEQ ID NOs:2-3. The resulting transgenic plant shows resistance to damage from pollen beetle and the target organism of the additional insecticidal dsRNA molecules.

### Example 6: Pip19 dsRNA in Insect Management

Prp19 dsRNA transgenes are combined with other dsRNA molecules in transgenic plants to provide redundant RNAi targeting and synergistic RNAi effects. Transgenic plants including, for example and without limitation, corn, soybean, and cotton expressing dsRNA that targets prp19 and other validated RNAi targets are useful for preventing feeding damage by insects.

Prp19 dsRNA transgenes are also combined in plants with Bacillus thuringiensis insecticidal protein technology and/or PIP-1 insecticidal polypeptides to represent new modes of action in Insect Resistance Management gene pyramids. A transgenic plant comprising a heterologous coding sequence in its genome that is transcribed into an iRNA molecule that targets pollen beetle prp19 is secondarily transformed via Agrobacterium or WHISKERS™ methodologies (see Petolino and Arnold (2009) Methods Mol. Biol. 526:59-67) to produce one or more insecticidal protein molecules, for example, Cry3, Cry34 and Cry35 insecticidal proteins. Plant transformation plasmid vectors are delivered via Agrobacterium or WHISKERS™-mediated transformation methods into suspension cells or immature embryos obtained from a plant comprising the heterologous coding sequence in its genome. Doubly-transformed plants are obtained that produce iRNA molecules and insecticidal proteins for control of insect pests. The resulting transgenic plants show synergistic protection against pollen beetle, due to the delayed onset of resistance to the control agents in pollen beetle populations infesting the plants

When prp19 iRNAs are combined with other dsRNA molecules that target insect pests and/or with insecticidal proteins in transgenic plants, a synergistic insecticidal effect is observed that also mitigates the development of resistant insect populations.

While the present disclosure may be susceptible to various modifications and alternative forms, specific embodiments have been described by way of example in detail herein. However, it should be understood that the present disclosure is not intended to be limited to the particular forms disclosed. Rather, the present disclosure is to cover all modifications, equivalents, and alternatives falling within the scope of the present disclosure as defined by the following appended claims and their legal equivalents.

Particular, non-limiting examples of representative embodiments are set forth below:

### Embodiment 1

An isolated nucleic acid molecule comprising at least one polynucleotide operably linked to a heterologous promoter, wherein the polynucleotide comprises any one or more of the nucleotide sequences selected from the group consisting of: SEQ ID NO:1; the complement of SEQ ID NO:1; the reverse complement of SEQ ID NO:1; the coding prp19 polynucleotide from Meligethes aeneus Fabricius comprising SEQ ID NOs:2-3; the complement of the codingprp19 polynucleotide from Meligethes aeneus Fabricius comprising SEQ ID NOs:2-3; the reverse complement of the coding prp19 polynucleotide from Meligethes aeneus Fabricius comprising SEQ ID NOs:2-3; a fragment of at least 15 contiguous nucleotides of the coding prp19 polynucleotide from Meligethes aeneus Fabricius comprising SEQ ID NOs:2-3; the complement of a fragment of at least 15 contiguous nucleotides of the coding prp19 polynucleotide from Meligethes aeneus Fabricius comprising SEQ ID NOs:2-3; the reverse complement of a fragment of at least 15 contiguous nucleotides of the coding prp19 polynucleotide from Meligethes aeneus Fabricius comprising SEQ ID NOs:2-3; a fragment of at least 19 contiguous nucleotides of the coding prp19 polynucleotide from Meligethes aeneus Fabricius comprising SEQ ID NOs:2-3; the complement of a fragment of at least 19 contiguous nucleotides of the coding prp19 polynucleotide from Meligethes aeneus Fabricius comprising SEQ ID NOs:2-3; the reverse complement of a fragment of at least 19 contiguous nucleotides of the coding prp19 polynucleotide from Meligethes aeneus Fabricius comprising SEQ ID NOs:2-3; a native coding sequence of a Meligethes organism comprising one or more of SEQ ID NOs:2-4; the complement of a native coding sequence of a Meligethes organism comprising one or more of SEQ ID NOs:2-4; the reverse complement of a native coding sequence of a Meligethes organism comprising one or more of SEQ ID NOs:2-4; a fragment of at least 15 contiguous nucleotides of a native coding sequence of a Meligethes organism comprising one or more of SEQ ID NOs:2-4; the complement of a fragment of at least 15 contiguous nucleotides of a native coding sequence of a Meligethes organism comprising one or more of SEQ ID NOs:2-4; the reverse complement of a fragment of at least 15 contiguous nucleotides of a native coding sequence of a Meligethes organism comprising one or more of SEQ ID NOs:2-4; a fragment of at least 19 contiguous nucleotides of a native coding sequence of a Meligethes organism comprising one or more of SEQ ID NOs:2-4; the complement of a fragment of at least 19 contiguous nucleotides of a native coding sequence of a Meligethes organism comprising one or more of SEQ ID NOs:2-4; the reverse complement of a fragment of at least 19 contiguous nucleotides of a native coding sequence of a Meligethes organism comprising one or more of SEQ ID NOs:2-4; SEQ ID NO:2; the complement of SEQ ID NO:2; the reverse complement of SEQ ID NO:2; SEQ ID NO:3; the complement of SEQ ID NO:3; the reverse complement of SEQ ID NO:3; SEQ ID NO:4; the complement of SEQ ID NO:4; the reverse complement of SEQ ID NO:4; a fragment of at least 15 or at least 19 contiguous nucleotides of any of SEQ ID NOs:2-4; the complement of a fragment of at least 15 or at least 19 contiguous nucleotides of any of SEQ ID NOs:2-4; and the reverse complement of a fragment of at least 15 or at least 19 contiguous nucleotides of any of SEQ ID NOs:2-4.

### Embodiment 2

The nucleic acid molecule of Embodiment 1, wherein the Meligethes organism is Meligethes aeneus Fabricius (Pollen Beetle).

### Embodiment 3

The nucleic acid molecule of either of Embodiments 1 and 2, wherein the nucleotide sequence is selected from the group consisting of SEQ ID NO:2, SEQ ID NO:3, SEQ ID NO:4, the complements of the foregoing, and the reverse complements of the foregoing.

### Embodiment 4

The nucleic acid molecule of any of Embodiments 1-3, wherein the molecule is a vector.

### Embodiment 5

A RNA molecule encoded by the nucleic acid molecule of any of Embodiments 1-4, wherein the RNA molecule comprises a polyribonucleotide encoded by the a nucleotide sequence comprised within the polynucleotide.

### Embodiment 6

The RNA molecule of Embodiment 5, wherein the molecule is a dsRNA molecule.

### Embodiment 7

The dsRNA molecule of Embodiment 6, wherein contacting the molecule with a coleopteran pest inhibits the expression of an endogenous nucleic acid molecule that is substantially complementary or reverse complementary to the polyribonucleotide.

### Embodiment 8

The dsRNA molecule of Embodiment 7, wherein the coleopteran pest is Meligethes aeneus Fabricius (Pollen Beetle).

### Embodiment 9

The dsRNA molecule of either of Embodiments 7 and 8, wherein contacting the molecule with the coleopteran pest kills or inhibits the growth and/or feeding of the pest.

### Embodiment 10

The dsRNA molecule of any of Embodiments 6-9, comprising a first, a second, and a third polyribonucleotide, wherein the first polyribonucleotide is encoded by the nucleotide sequence, wherein the third polyribonucleotide is linked to the first polyribonucleotide by the second polyribonucleotide, and wherein the third polyribonucleotide is substantially the reverse complement of the first polyribonucleotide, such that the first and the third polyribonucleotides hybridize when transcribed into a ribonucleic acid to form the dsRNA.

### Embodiment 11

The dsRNA molecule of any of Embodiments 6-9, wherein the molecule comprises a single-stranded polyribonucleotide of between about 19 and about 30 nucleotides in length that is encoded by the nucleotide sequence.

### Embodiment 12

The vector of Embodiment 4, wherein the heterologous promoter is functional in a plant cell, and wherein the vector is a plant transformation vector or plant expression vector.

### Embodiment 13

A cell comprising the nucleic acid molecule of any of Embodiments 1-12.

### Embodiment 14

The cell of Embodiment 13, wherein the cell is a prokaryotic cell.

### Embodiment 15

The cell of Embodiment 13, wherein the cell is a eukaryotic cell.

### Embodiment 16

The cell of Embodiment 15, wherein the cell is a plant cell.

### Embodiment 17

A plant part comprising the plant cell of Embodiment 16 or the nucleic acid molecule of any of Embodiments 1-12.

### Embodiment 18

The plant part of Embodiment 17, wherein the plant part is a seed.

### Embodiment 19

A transgenic plant comprising the plant part of either of Embodiments 17 and 18, or the plant cell of Embodiment 16.

### Embodiment 20

A food product or commodity product produced from the plant of Embodiment 19 or the plant part of either of Embodiments 17 and 18, wherein the product comprises a detectable amount of the nucleic acid molecule.

### Embodiment 21

The food product or commodity product of Embodiment 20, wherein the product is selected from an oil, meal, and a fiber.

### Embodiment 22

The plant cell of Embodiment 17, the plant part of either of Embodiments 17 and 18, or the plant of Embodiment 19, comprising the dsRNA molecule of any of Embodiments 6-11.

### Embodiment 23

The plant cell, plant part, or plant of Embodiment 22, wherein the plant is Zea mays, Glycine max, a Brassica sp., a Gossypium sp., or Poaceae.

### Embodiment 24

The plant cell, plant part, or plant of Embodiment 23, wherein the plant is a Brassica sp.

### Embodiment 25

The plant cell, plant part, or plant of Embodiment 24, wherein the plant is canola.

### Embodiment 26

The plant cell, plant part, or plant of any of Embodiments 22-25, wherein the a dsRNA molecule inhibits the expression of an endogenous polynucleotide that is specifically complementary or reverse complementary to a polyribonucleotide comprised in the RNA molecule when an insect pest ingests a part of the plant.

### Embodiment 27

The plant cell, plant part, or plant of Embodiment 26, wherein the coleopteran pest is Meligethes aeneus Fabricius (Pollen Beetle).

### Embodiment 28

A sprayable formulation or bait composition comprising the RNA molecule of any of Embodiments 5-11.

### Embodiment 29

The nucleic acid molecule of any of Embodiments 1-4, further comprising at least one additional polynucleotide operably linked to a heterologous promoter, wherein the additional polynucleotide encodes an iRNA molecule.

### Embodiment 30

A method for controlling an insect pest population, the method comprising contacting an insect pest of the population with an agent comprising a dsRNA molecule that functions upon contact with the insect pest to inhibit a biological function within the pest, wherein the molecule comprises a polyribonucleotide that is specifically hybridizable with a reference polyribonucleotide selected from the group consisting of any of SEQ ID NOs:12-15; the complement of any of SEQ ID NOs:12-15; the reverse complement of any of SEQ ID NOs:12-15; a fragment of at least 15 or at least 19 contiguous nucleotides of any of SEQ ID NOs:13-15; the complement of a fragment of at least 15 or at least 19 contiguous nucleotides of any of SEQ ID NOs:13-15; the reverse complement of a fragment of at least 15 or at least 19 contiguous nucleotides of any of SEQ ID NOs:13-15; all or a fragment of at least 15 or at least 19 contiguous nucleotides of a transcript of the PBprp19 gene comprising SEQ ID NOs:2-3; the complement of all or a fragment of at least 15 or at least 19 contiguous nucleotides of a transcript of the PB prp19 gene comprising SEQ ID NOs:2-3; and the reverse complement of all or a fragment of at least 15 or at least 19 contiguous nucleotides of a transcript of the PBprp19 gene comprising SEQ ID NOs:2-3.

### Embodiment 31

The method according to Embodiment 30, wherein the polyribonucleotide is specifically hybridizable with a reference polyribonucleotide selected from the group consisting of any of SEQ ID NOs:13-15; the complement of any of SEQ ID NOs:13-15; the reverse complement of any of SEQ ID NOs:13-15; a fragment of at least 15 or at least 19 contiguous nucleotides of any of SEQ ID NOs:13-15; the complement of a fragment of at least 15 or at least 19 contiguous nucleotides of any of SEQ ID NOs:13-15; and the reverse complement of a fragment of at least 15 or at least 19 contiguous nucleotides of any of SEQ ID NOs:13-15.

### Embodiment 32

A method for controlling an insect pest population, the method comprising contacting an insect pest of the population with an agent comprising a dsRNA molecule comprising a first and a second polyribonucleotide, wherein the dsRNA molecule functions upon contact with the insect pest to inhibit a biological function within the insect pest, wherein the first polyribonucleotide comprises a nucleotide sequence having from about 90% to about 100% sequence identity to from about 15 or about 19 to about 30 contiguous nucleotides of the reference polyribonucleotide encoded by the PBprp19 gene comprising SEQ ID NOs:2-3, and wherein the first polyribonucleotide is specifically hybridized to the second polyribonucleotide.

### Embodiment 33

The method according to Embodiment 32, wherein the reference polyribonucleotide is any of SEQ ID NOs:13-15.

### Embodiment 34

The method according to any of Embodiments 30-33, wherein contacting the pest with the agent comprises contacting the pest with a sprayable formulation comprising the dsRNA molecule.

### Embodiment 35

The method according to any of Embodiments 30-33, wherein contacting the pest with the agent comprises feeding the pest with the agent, and the agent is a plant cell comprising the dsRNA molecule or an RNA bait comprising the dsRNA molecule.

### Embodiment 36

A method for controlling an insect pest population, the method comprising providing in a host plant of the insect pest a plant cell comprising the nucleic acid molecule of any of Embodiments 1-4, wherein the polynucleotide is expressed to produce a RNA molecule that functions upon contact with an insect pest belonging to the population to inhibit the expression of a target sequence within the insect pest and results in decreased growth and/or survival of the insect pest or pest population, relative to development of the same pest species on a plant of the same host plant species that does not comprise the polynucleotide.

### Embodiment 37

The method according to Embodiment 36, wherein the insect pest population is reduced relative to a population of the same pest species infesting a host plant of the same host plant species lacking a plant cell comprising the nucleic acid molecule.

### Embodiment 38

A method of controlling an insect pest infestation in a plant, the method comprising providing in the diet of the insect pest an RNA molecule comprising a polyribonucleotide that is specifically hybridizable with a reference polyribonucleotide selected from the group consisting of: the PB mRNA comprising SEQ ID NOs:13-15; the complement of the PB mRNA comprising SEQ ID NOs:13-15; the reverse complement of the PB mRNA comprising SEQ ID NOs:13-15; SEQ ID NOs:13-15; the complement of any of SEQ ID NOs:13-15; the reverse complement of any of SEQ ID NOs:13-15; a fragment of at least 15 or at least 19 contiguous nucleotides of any SEQ ID NOs:13-15; the complement of a fragment of at least 15 or at least 19 contiguous nucleotides of any of SEQ ID NOs:13-15; and the reverse complement of a fragment of at least 15 or at least 19 contiguous nucleotides of any of SEQ ID NOs:13-15.

### Embodiment 39

The method according to Embodiment 38, wherein the diet comprises a plant cell comprising a polynucleotide that is transcribed to express the RNA molecule.

### Embodiment 40

A method for improving the yield of a crop, the method comprising cultivating in the crop a plant comprising the nucleic acid molecule of any of Embodiments 1-4 to allow the expression of the polynucleotide.

### Embodiment 41

The method according to Embodiment 40, wherein expression of the polynucleotide produces a dsRNA molecule that suppresses at least a first target gene in an insect pest that has contacted a portion of the plant, thereby inhibiting the development or growth of the insect pest and loss of yield due to infection by the insect pest.

### Embodiment 42

A method for producing a transgenic plant cell, the method comprising transforming a plant cell with the vector of Embodiment 12; culturing the transformed plant cell under conditions sufficient to allow for development of a plant cell culture comprising a plurality of transgenic plant cells; selecting for transgenic plant cells that have integrated the polynucleotide into their genomes; screening the transgenic plant cells for expression of a dsRNA molecule encoded by the polynucleotide; and selecting a transgenic plant cell that expresses the dsRNA.

### Embodiment 43

A method for producing an insect pest-resistant transgenic plant, the method comprising regenerating a transgenic plant from a transgenic plant cell comprising the nucleic acid molecule of any of Embodiments 1-4, wherein expression of a dsRNA molecule encoded by the polynucleotide is sufficient to reduce the expression of a target gene in the insect pest when it contacts the RNA molecule.

### Embodiment 44

The method according to any of Embodiments 30-39, 41, and 43, wherein the insect pest is a coleopteran pest.

### Embodiment 45

The method according to Embodiment 44, wherein the coleopteran pest is Meligethes aeneus Fabricius (Pollen Beetle).

### Embodiment 46

The method according to any of Embodiments 35-37 and 39-43, wherein the plant or plant cell is Zea mays, Glycine max, Brassica sp., Gossypium sp., or a plant or plant cell of the family Poaceae.

### Embodiment 47

The method according to Embodiment 46, wherein the plant or plant cell is a Brassica sp.

### Embodiment 48

The method according to Embodiment 47, wherein the plant or plant cell is canola.

### Embodiment 49

The nucleic acid molecule of any of Embodiments 1-4, further comprising a polynucleotide encoding an insecticidal polypeptide from Bacillus thuringiensis.

### Embodiment 50

The plant cell, plant part, or plant of any of Embodiments 22-27, further comprising a polynucleotide encoding an insecticidal polypeptide from Bacillus thuringiensis, Alcaligenes spp., or Pseudomonas spp.

### Embodiment 51

The method according to any of Embodiments 35-37 and 39-48, wherein the plant or plant cell comprises a polynucleotide encoding an insecticidal polypeptide from Bacillus thuringiensis, Alcaligenes spp., or Pseudomonas spp.

### Embodiment 52

The nucleic acid molecule of Embodiment 49, the plant cell, plant part, or plant of Embodiment 50, or the method according to Embodiment 51, wherein the insecticidal polypeptide is selected from the group of B. thuringiensis insecticidal polypeptides consisting of Cry1B, Cry1I, Cry3, Cry7A, Cry8, Cry9D, Cry14, Cry18, Cry22, Cry23, Cry34, Cry35, Cry36, Cry37, Cry43, Cry55, Cyt1A, and Cyt2C.

1. An isolated nucleic acid molecule comprising at least one polynucleotide operably linked to a heterologous promoter, wherein the polynucleotide comprises a nucleotide sequence selected from the group consisting of:
SEQ ID NO:1; the complement or reverse complement of SEQ ID NO:1; a fragment of at least 15 contiguous nucleotides of the endogenous coding polynucleotide from Meligethes aeneus Fabricius comprising SEQ ID NOs:2-3; the complement or reverse complement of a fragment of at least 15 contiguous nucleotides of the endogenous coding polynucleotide from Meligethes aeneus Fabricius comprising SEQ ID NOs:2-3; a native coding sequence of a Meligethes organism comprising SEQ ID NO:4; the complement or reverse complement of a native coding sequence of a Meligethes organism comprising SEQ ID NO:4; a fragment of at least 15 contiguous nucleotides of a native coding sequence of a Meligethes organism comprising SEQ ID NO:4; and the complement or reverse complement of a fragment of at least 15 contiguous nucleotides of a native coding sequence of a Meligethes organism comprising SEQ ID NO:4.

2. The nucleic acid molecule of claim 1, wherein the nucleotide sequence is selected from the group consisting of SEQ ID NOs:2-4; a fragment of at least 15 contiguous nucleotides of any of SEQ ID NOs:2-4; and the complements and reverse complements of the foregoing.

3. The nucleic acid molecule of claim 1, wherein the molecule is a vector.

4. An isolated nucleic acid molecule characterized by a polynucleotide operably linked to a heterologous promoter, wherein the polynucleotide is SEQ ID NO:4; the complement of SEQ ID NO:4, or the reverse complement of SEQ ID NO:4.

5. A ribonucleic acid (RNA) molecule encoded by the nucleic acid molecule of claim 1, wherein the RNA molecule comprises a polyribonucleotide encoded by the nucleotide sequence.

6. The RNA molecule of claim 5, wherein the molecule is a double-stranded ribonucleic acid (dsRNA) molecule.

7. The dsRNA molecule of claim 6, wherein contacting the polyribonucleotide with an insect pest inhibits the expression of an endogenous nucleic acid molecule that is specifically complementary to the polyribonucleotide.

8. The dsRNA molecule of claim 7, wherein contacting the polyribonucleotide with the insect pest kills or inhibits the growth and/or feeding of the pest.

9. The dsRNA of claim 6, comprising a first, a second, and a third polyribonucleotide, wherein the first polyribonucleotide is transcribed from the polynucleotide, wherein the third polyribonucleotide is linked to the first polyribonucleotide by the second polyribonucleotide, and wherein the third polyribonucleotide is substantially the reverse complement of the first polyribonucleotide, such that the first and the third polyribonucleotides hybridize when transcribed into a ribonucleic acid to form the dsRNA.

10. The dsRNA of claim 6, wherein the molecule comprises a first and a second polyribonucleotide, wherein the first polyribonucleotide is transcribed from the polynucleotide, wherein the third polyribonucleotide is a separate strand from the second polyribonucleotide, and wherein the first and the second polyribonucleotides hybridize to form the dsRNA.

11. The vector of claim 3, wherein the vector is a plant transformation vector, and wherein the heterologous promoter is functional in a plant cell.

12. A cell comprising the nucleic acid molecule of claim 1.

13. The cell of claim 12, wherein the cell is a prokaryotic cell.

14. The cell of claim 12, wherein the cell is a eukaryotic cell.

15. The cell of claim 14, wherein the cell is a plant cell.

16. A plant comprising the nucleic acid molecule of claim 1.

17. A part of the plant of claim 16, wherein the plant part comprises the nucleic acid molecule.

18. The plant part of claim 17, wherein the plant part is a seed.

19. A food product or commodity product produced from the plant of claim 16, wherein the product comprises a detectable amount of the polynucleotide.

20. The plant of claim 16, wherein the polynucleotide is expressed in the plant as a double-stranded ribonucleic acid (dsRNA) molecule.

21. The plant cell of claim 15, wherein the cell is a cell from a Brassica plant species.

22. The plant of claim 16, wherein the plant is a Brassica plant species.

23. The plant of claim 16, wherein the polynucleotide is expressed in the plant as a double-stranded ribonucleic acid (dsRNA) molecule, and the dsRNA molecule inhibits the expression of an endogenous polynucleotide that is specifically complementary to the RNA molecule when an insect pest ingests a part of the plant.

24. The nucleic acid molecule of claim 1, further comprising at least one additional polynucleotide operably linked to a heterologous promoter, wherein the additional polynucleotide encodes an RNA molecule.

25. The nucleic acid molecule of claim 24, wherein the molecule is a plant transformation vector, and wherein the heterologous promoter is functional in a plant cell.

26. A method for controlling an insect pest population, the method comprising providing an agent comprising a ribonucleic acid (RNA) molecule that functions upon contact with the insect pest to inhibit a biological function within the pest, wherein the RNA molecule comprises a polyribonucleotide that is specifically hybridizable with a target polyribonucleotide selected from the group consisting of SEQ ID NOs:12-15; the complement of any of SEQ ID NOs:12-15; the reverse complement of any of SEQ ID NOs:12-15; a fragment of at least 15 contiguous nucleotides of any of SEQ ID NOs:13-15; the complement of a fragment of at least 15 contiguous nucleotides of any of SEQ ID NOs:13-15; the reverse complement of a fragment of at least 15 contiguous nucleotides of any of SEQ ID NOs:13-15; a transcript of the PB prp19 coding polynucleotide comprising SEQ ID NOs:2-3; the complement of a transcript of the PB prp19 coding polynucleotide comprising SEQ ID NOs:2-3; the reverse complement of a transcript of the PB prp19 coding polynucleotide comprising SEQ ID NOs:2-3; a fragment of at least 15 contiguous nucleotides of a transcript of the PB prp19 coding polynucleotide comprising SEQ ID NOs:2-3; the complement of a fragment of at least 15 contiguous nucleotides of a transcript of the PB prp19 coding polynucleotide comprising SEQ ID NOs:2-3; and the reverse complement of a fragment of at least 15 contiguous nucleotides of a transcript of the PB prp19 coding polynucleotide comprising SEQ ID NOs:2-3.

27. The method according to claim 26, wherein the RNA molecule is a double-stranded RNA (dsRNA) molecule.

28. The method according to claim 27, wherein providing the agent comprises contacting the insect pest with a sprayable composition comprising the agent or feeding the insect pest with an RNA bait comprising the agent.

29. The method according to claim 27, wherein providing the agent is a transgenic plant cell expressing the dsRNA molecule.

30. A method for controlling an insect pest population, the method comprising:
providing an agent comprising a first and a second polyribonucleotide that functions upon contact with an insect pest to inhibit a biological function within the insect pest, wherein the first polyribonucleotide comprises a nucleotide sequence having from about 90% to about 100% sequence identity to from about 15 to about 30 contiguous nucleotides of a polyribonucleotide selected from the group consisting of SEQ ID NOs:13-15, and wherein the first polyribonucleotide is specifically hybridized to the second polyribonucleotide.

31. A method for controlling an insect pest population, the method comprising:
providing in a host plant of an insect pest a plant cell comprising the nucleic acid molecule of claim 1, wherein the polynucleotide is expressed to produce a double-stranded ribonucleic acid (dsRNA) molecule that functions upon contact with an insect pest belonging to the population to inhibit the expression of a target sequence within the insect pest and results in decreased growth and/or survival of the insect pest or pest population, relative to development of the same pest species on a plant of the same host plant species that does not comprise the polynucleotide.

32. The method according to claim 31, wherein the insect pest population is reduced relative to a population of the same pest species infesting a host plant of the same host plant species lacking a plant cell comprising the nucleic acid molecule.

33. A method of controlling an insect pest infestation in a plant, the method comprising providing in the diet of the insect pest a ribonucleic acid (RNA) molecule comprising a polyribonucleotide that is specifically hybridizable with a reference polyribonucleotide selected from the group consisting of:
SEQ ID NOs:12-15;
the complement or reverse complement of any of SEQ ID NOs:12-15;
a fragment of at least 15 contiguous nucleotides of any of SEQ ID NOs:13-15;
the complement or reverse complement of a fragment of at least 15 contiguous nucleotides of any of SEQ ID NOs:13-15;
a transcript of the PB prp19 coding polynucleotide comprising SEQ ID NOs:2-3;
the complement or reverse complement of a transcript of the PB prp19 coding polynucleotide comprising SEQ ID NOs:2-3;
a fragment of at least 15 contiguous nucleotides of a transcript of the PB prp19 coding polynucleotide comprising SEQ ID NOs:2-3; and
the complement or reverse complement of a fragment of at least 15 contiguous nucleotides of a transcript of the PB prp19 coding polynucleotide comprising SEQ ID NOs:2-3.

34. The method according to claim 33, wherein the RNA molecule is a double-stranded RNA (dsRNA) molecule.

35. The method according to claim 34, wherein the diet comprises a plant cell comprising a polynucleotide that is transcribed to express the dsRNA molecule.

36. A method for improving the yield of a crop, the method comprising:
cultivating in the crop a plant comprising the nucleic acid of claim 1 to allow the expression of the polynucleotide.

37. The method according to claim 36, wherein the plant is a Brassica species.

38. The method according to claim 36, wherein expression of the polynucleotide produces a double-stranded RNA (dsRNA) molecule that suppresses a target gene in an insect pest that has contacted a portion of the plant, thereby inhibiting the development or growth of the insect pest and loss of yield due to infection by the insect pest.

39. A method for producing a transgenic plant cell, the method comprising:
transforming a plant cell with the plant transformation vector of claim 11;
culturing the transformed plant cell under conditions sufficient to allow for development of a plant cell culture comprising a plurality of transgenic plant cells;
selecting for transgenic plant cells that have integrated the polynucleotide into their genomes;
screening the transgenic plant cells for expression of a double-stranded ribonucleic acid (dsRNA) molecule encoded by the polynucleotide; and
selecting a transgenic plant cell that expresses the dsRNA.

40. A method for producing an insect pest-resistant transgenic plant, the method comprising:
regenerating a transgenic plant from a transgenic plant cell comprising the nucleic acid molecule of claim 1, wherein expression of a double-stranded ribonucleic acid (dsRNA) molecule encoded by the polynucleotide is sufficient to modulate the expression of a target gene in the insect pest when it contacts the RNA molecule.

41. A method for producing a transgenic plant cell, the method comprising:
transforming a plant cell with a vector comprising a means for providing prp19-mediated Meligethes pest protection to a plant;
culturing the transformed plant cell under conditions sufficient to allow for development of a plant cell culture comprising a plurality of transformed plant cells;
selecting for transformed plant cells that have integrated the means for providing prp19-mediated Meligethes pest protection to a plant into their genomes;
screening the transformed plant cells for expression of a means for inhibiting expression of a prp19 gene in a Meligethes pest; and
selecting a plant cell that expresses the means for inhibiting expression of a prp19 gene in a Meligethes pest.

42. A method for producing a transgenic plant, the method comprising:
regenerating a transgenic plant from the transgenic plant cell produced by the method according to claim 41, wherein plant cells of the plant comprise the means for inhibiting expression of a prp19 gene in a Meligethes pest.

43. The method according to claim 42, wherein expression of the means for inhibiting expression of a prp19 gene in a Meligethes pest is sufficient to reduce the expression of a target prp19 gene in a Meligethes pest that infests the transgenic plant.

44. A plant comprising means for inhibiting expression of a prp19 gene in a Meligethes pest.

45. The nucleic acid of claim 1, further comprising a polynucleotide encoding an insecticidal polypeptide from Bacillus thuringiensis, Alcaligenes spp., or Pseudomonas spp.

46. The nucleic acid of claim 45, wherein the insecticidal polypeptide is selected from the group of B. thuringiensis insecticidal polypeptides consisting of Cry1B, Cry 1I, Cry2A, Cry3, Cry7A, Cry8, Cry9D, Cry14, Cry18, Cry22, Cry23, Cry34, Cry35, Cry36, Cry37, Cry43, Cry55, Cyt1A, and Cyt2C.

47. The plant cell of claim 15, wherein the cell comprises a polynucleotide encoding an insecticidal polypeptide from Bacillus thuringiensis, Alcaligenes spp., or Pseudomonas spp.

48. The plant cell of claim 47, wherein the insecticidal polypeptide is selected from the group of B. thuringiensis insecticidal polypeptides consisting of Cry1B, Cry1I, Cry3, Cry7A, Cry8, Cry9D, Cry14, Cry18, Cry22, Cry23, Cry34, Cry35, Cry36, Cry37, Cry43, Cry55, Cyt1A, and Cyt2C.

49. The plant of claim 16, wherein the plant comprises a polynucleotide encoding an insecticidal polypeptide from Bacillus thuringiensis, Alcaligenes spp., or Pseudomonas spp.

50. The plant of claim 49, wherein the insecticidal polypeptide is selected from the group of B. thuringiensis insecticidal polypeptides consisting of Cry1B, Cry 1I, Cry2A, Cry3, Cry7A, Cry8, Cry9D, Cry14, Cry18, Cry22, Cry23, Cry34, Cry35, Cry36, Cry37, Cry43, Cry55, Cyt1A, and Cyt2C.

51. The method according to claim 31, wherein the plant cell comprises a polynucleotide encoding an insecticidal polypeptide from Bacillus thuringiensis, Alcaligenes spp., or Pseudomonas spp.

52. The method according to claim 51, wherein the insecticidal polypeptide is selected from the group of B. thuringiensis insecticidal polypeptides consisting of Cry1B, Cry 1I, Cry2A, Cry3, Cry7A, Cry8, Cry9D, Cry14, Cry18, Cry22, Cry23, Cry34, Cry35, Cry36, Cry37, Cry43, Cry55, Cyt1A, and Cyt2C.


SEQUENCE LISTING (31 sequences)

SEQ ID NO: 1 - DNA, 1400 bp, Meligethes aeneus
  misc_feature (895)..(927): n is a, c, g, or t
  misc_feature (1368)..(1400): n is a, c, g, or t
atggcgttgtgttgtgccatttcgaacgaagttccggagcatccagtggtttctccttcatccggagtaatattcgagagaagaataatcgaaaaatacatacaggagaatggagtggaccctataagtggaaaagaagtcgctatagatgaattaatagaaatcaaaacgcctccaattgtaaaaccaaagccaccaagtgctactagcatcccagctacattaaaactactgcaggatgaatgggatgcggttatgctgtacagttttacacaacggcagcaattacaaacagcgcgacaggaattatcgcacgctttatatcaacacgatgctgcctgcagggtgattgcaaggttaaacaaggaagtaactgctgcaagagaggctctggcaacattgaaaccacaagctggtattactacagtccctcaacctgctgttgctgctgaagctggcggtgttgccaatcaacccactgaacaagctggcatgagtgtggaggttatacaaaaattgcaagataaggcagccgttctgacacaggagcgtaaaaagaggggtcgcacagttccagaagagttgactactcaggaacagttgaggagtttcaggacattggcatcccttattggtcttcattccgccagtattcctggtattttagctttagatgtgcacagttcagataccagtaaagtattaacaggaggaaacgacaaaaatgctactgtattcaacaaagataccgaacaggtcgtcacaattttaaaaggacacactaaaaaagtgacaaaggtcatttaccatcccgaagaagatattgttattacagcttctcccgattcgacaatcagagtttggaacgtgccgacctcgcaaaccactctcctcnnnnnnnnnnnnnnnnnnnnnnnnnnnnnnnnntccctgcaccccacgggagactacgttctgtccacttccgaagaccaacattgggcgttttccgacatccgtaccggcagacttctgaccaaagtttccgatcaatcgaacgttccgctgaccaccgcgcagctgcatcccgacggtctcatcttcggcaccggaaccggggattcccaggtcaaaatttgggatttgaaggagcagagcaacgtcgccaacttcacaggacattccggagcaatcaccacgatttccttctcggaaaacgggtattacttggccactgccgccgacgacgcgtgcgtaaaactatgggatttgcgtaaattaagaaacttcaaaactctacaactcgacgaaggttaccaaatcaaagatttgtgtttcgatcaaagcggaacctatttggccgttgccggaactgacgtcagggtttannnnnnnnnnnnnnnnnnnnnnnnnnnnnnnnn

SEQ ID NO: 2 - DNA, 894 bp, Meligethes aeneus
atggcgttgtgttgtgccatttcgaacgaagttccggagcatccagtggtttctccttcatccggagtaatattcgagagaagaataatcgaaaaatacatacaggagaatggagtggaccctataagtggaaaagaagtcgctatagatgaattaatagaaatcaaaacgcctccaattgtaaaaccaaagccaccaagtgctactagcatcccagctacattaaaactactgcaggatgaatgggatgcggttatgctgtacagttttacacaacggcagcaattacaaacagcgcgacaggaattatcgcacgctttatatcaacacgatgctgcctgcagggtgattgcaaggttaaacaaggaagtaactgctgcaagagaggctctggcaacattgaaaccacaagctggtattactacagtccctcaacctgctgttgctgctgaagctggcggtgttgccaatcaacccactgaacaagctggcatgagtgtggaggttatacaaaaattgcaagataaggcagccgttctgacacaggagcgtaaaaagaggggtcgcacagttccagaagagttgactactcaggaacagttgaggagtttcaggacattggcatcccttattggtcttcattccgccagtattcctggtattttagctttagatgtgcacagttcagataccagtaaagtattaacaggaggaaacgacaaaaatgctactgtattcaacaaagataccgaacaggtcgtcacaattttaaaaggacacactaaaaaagtgacaaaggtcatttaccatcccgaagaagatattgttattacagcttctcccgattcgacaatcagagtttggaacgtgccgacctcgcaaaccactctcctc

SEQ ID NO: 3 - DNA, 440 bp, Meligethes aeneus
tccctgcaccccacgggagactacgttctgtccacttccgaagaccaacattgggcgttttccgacatccgtaccggcagacttctgaccaaagtttccgatcaatcgaacgttccgctgaccaccgcgcagctgcatcccgacggtctcatcttcggcaccggaaccggggattcccaggtcaaaatttgggatttgaaggagcagagcaacgtcgccaacttcacaggacattccggagcaatcaccacgatttccttctcggaaaacgggtattacttggccactgccgccgacgacgcgtgcgtaaaactatgggatttgcgtaaattaagaaacttcaaaactctacaactcgacgaaggttaccaaatcaaagatttgtgtttcgatcaaagcggaacctatttggccgttgccggaactgacgtcagggttta

SEQ ID NO: 4 - DNA, 306 bp, Meligethes aeneus
ggcgttgtgttgtgccatttcgaacgaagttccggagcatccagtggtttctccttcatccggagtaatattcgagagaagaataatcgaaaaatacatacaggagaatggagtggaccctataagtggaaaagaagtcgctatagatgaattaatagaaatcaaaacgcctccaattgtaaaaccaaagccaccaagtgctactagcatcccagctacattaaaactactgcaggatgaatgggatgcggttatgctgtacagttttacacaacggcagcaattacaaacagcgcgacaggaatt

SEQ ID NO: 5 - protein, 467 aa, Meligethes aeneus
  misc_feature (299)..(309): Xaa can be any naturally occurring amino acid
  misc_feature (456)..(467): Xaa can be any naturally occurring amino acid
MALCCAISNEVPEHPVVSPSSGVIFERRIIEKYIQENGVDPISGKEVAIDELIEIKTPPIVKPKPPSATSIPATLKLLQDEWDAVMLYSFTQRQQLQTARQELSHALYQHDAACRVIARLNKEVTAAREALATLKPQAGITTVPQPAVAAEAGGVANQPTEQAGMSVEVIQKLQDKAAVLTQERKKRGRTVPEELTTQEQLRSFRTLASLIGLHSASIPGILALDVHSSDTSKVLTGGNDKNATVFNKDTEQVVTILKGHTKKVTKVIYHPEEDIVITASPDSTIRVWNVPTSQTTLLXXXXXXXXXXXSLHPTGDYVLSTSEDQHWAFSDIRTGRLLTKVSDQSNVPLTTAQLHPDGLIFGTGTGDSQVKIWDLKEQSNVANFTGHSGAITTISFSENGYYLATAADDACVKLWDLRKLRNFKTLQLDEGYQIKDLCFDQSGTYLAVAGTDVRVXXXXXXXXXXXX

SEQ ID NO: 6 - protein, 298 aa, Meligethes aeneus
MALCCAISNEVPEHPVVSPSSGVIFERRIIEKYIQENGVDPISGKEVAIDELIEIKTPPIVKPKPPSATSIPATLKLLQDEWDAVMLYSFTQRQQLQTARQELSHALYQHDAACRVIARLNKEVTAAREALATLKPQAGITTVPQPAVAAEAGGVANQPTEQAGMSVEVIQKLQDKAAVLTQERKKRGRTVPEELTTQEQLRSFRTLASLIGLHSASIPGILALDVHSSDTSKVLTGGNDKNATVFNKDTEQVVTILKGHTKKVTKVIYHPEEDIVITASPDSTIRVWNVPTSQTTLL

SEQ ID NO: 7 - protein, 146 aa, Meligethes aeneus
SLHPTGDYVLSTSEDQHWAFSDIRTGRLLTKVSDQSNVPLTTAQLHPDGLIFGTGTGDSQVKIWDLKEQSNVANFTGHSGAITTISFSENGYYLATAADDACVKLWDLRKLRNFKTLQLDEGYQIKDLCFDQSGTYLAVAGTDVRV

SEQ ID NO: 8 - DNA, 24 bp, Artificial Sequence
  T7 promoter oligonucleotide
ttaatacgactcactatagggaga

SEQ ID NO: 9 - DNA, 43 bp, Artificial Sequence
  Primer prp19 reg1_For
taatacgactcactatagggagaggcgttgtgttgtgccattt

SEQ ID NO: 10 - DNA, 43 bp, Artificial Sequence
  Primer prp19 reg1_Rev
taatacgactcactatagggagaaattcctgtcgcgctgtttg

SEQ ID NO: 11 - DNA, 837 bp, Artificial Sequence
  prp19 reg1 hpRNA-forming RNA
ggcgttgtgttgtgccatttcgaacgaagttccggagcatccagtggtttctccttcatccggagtaatattcgagagaagaataatcgaaaaatacatacaggagaatggagtggaccctataagtggaaaagaagtcgctatagatgaattaatagaaatcaaaacgcctccaattgtaaaaccaaagccaccaagtgctactagcatcccagctacattaaaactactgcaggatgaatgggatgcggttatgctgtacagttttacacaacggcagcaattacaaacagcgcgacaggaattgactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttaaattcctgtcgcgctgtttgtaattgctgccgttgtgtaaaactgtacagcataaccgcatcccattcatcctgcagtagttttaatgtagctgggatgctagtagcacttggtggctttggttttacaattggaggcgttttgatttctattaattcatctatagcgacttcttttccacttatagggtccactccattctcctgtatgtatttttcgattattcttctctcgaatattactccggatgaaggagaaaccactggatgctccggaacttcgttcgaaatggcacaacacaacgcc

SEQ ID NO: 12 - RNA, 1400 nt, Meligethes aeneus
  misc_feature (895)..(927): n is a, c, g, or u
  misc_feature (1368)..(1400): n is a, c, g, or u
auggcguuguguugugccauuucgaacgaaguuccggagcauccagugguuucuccuucauccggaguaauauucgagagaagaauaaucgaaaaauacauacaggagaauggaguggacccuauaaguggaaaagaagucgcuauagaugaauuaauagaaaucaaaacgccuccaauuguaaaaccaaagccaccaagugcuacuagcaucccagcuacauuaaaacuacugcaggaugaaugggaugcgguuaugcuguacaguuuuacacaacggcagcaauuacaaacagcgcgacaggaauuaucgcacgcuuuauaucaacacgaugcugccugcagggugauugcaagguuaaacaaggaaguaacugcugcaagagaggcucuggcaacauugaaaccacaagcugguauuacuacagucccucaaccugcuguugcugcugaagcuggcgguguugccaaucaacccacugaacaagcuggcaugaguguggagguuauacaaaaauugcaagauaaggcagccguucugacacaggagcguaaaaagaggggucgcacaguuccagaagaguugacuacucaggaacaguugaggaguuucaggacauuggcaucccuuauuggucuucauuccgccaguauuccugguauuuuagcuuuagaugugcacaguucagauaccaguaaaguauuaacaggaggaaacgacaaaaaugcuacuguauucaacaaagauaccgaacaggucgucacaauuuuaaaaggacacacuaaaaaagugacaaaggucauuuaccaucccgaagaagauauuguuauuacagcuucucccgauucgacaaucagaguuuggaacgugccgaccucgcaaaccacucuccucnnnnnnnnnnnnnnnnnnnnnnnnnnnnnnnnnucccugcaccccacgggagacuacguucuguccacuuccgaagaccaacauugggcguuuuccgacauccguaccggcagacuucugaccaaaguuuccgaucaaucgaacguuccgcugaccaccgcgcagcugcaucccgacggucucaucuucggcaccggaaccggggauucccaggucaaaauuugggauuugaaggagcagagcaacgucgccaacuucacaggacauuccggagcaaucaccacgauuuccuucucggaaaacggguauuacuuggccacugccgccgacgacgcgugcguaaaacuaugggauuugcguaaauuaagaaacuucaaaacucuacaacucgacgaagguuaccaaaucaaagauuuguguuucgaucaaagcggaaccuauuuggccguugccggaacugacgucaggguuuannnnnnnnnnnnnnnnnnnnnnnnnnnnnnnnn

SEQ ID NO: 13 - RNA, 894 nt, Meligethes aeneus
auggcguuguguugugccauuucgaacgaaguuccggagcauccagugguuucuccuucauccggaguaauauucgagagaagaauaaucgaaaaauacauacaggagaauggaguggacccuauaaguggaaaagaagucgcuauagaugaauuaauagaaaucaaaacgccuccaauuguaaaaccaaagccaccaagugcuacuagcaucccagcuacauuaaaacuacugcaggaugaaugggaugcgguuaugcuguacaguuuuacacaacggcagcaauuacaaacagcgcgacaggaauuaucgcacgcuuuauaucaacacgaugcugccugcagggugauugcaagguuaaacaaggaaguaacugcugcaagagaggcucuggcaacauugaaaccacaagcugguauuacuacagucccucaaccugcuguugcugcugaagcuggcgguguugccaaucaacccacugaacaagcuggcaugaguguggagguuauacaaaaauugcaagauaaggcagccguucugacacaggagcguaaaaagaggggucgcacaguuccagaagaguugacuacucaggaacaguugaggaguuucaggacauuggcaucccuuauuggucuucauuccgccaguauuccugguauuuuagcuuuagaugugcacaguucagauaccaguaaaguauuaacaggaggaaacgacaaaaaugcuacuguauucaacaaagauaccgaacaggucgucacaauuuuaaaaggacacacuaaaaaagugacaaaggucauuuaccaucccgaagaagauauuguuauuacagcuucucccgauucgacaaucagaguuuggaacgugccgaccucgcaaaccacucuccuc

SEQ ID NO: 14 - RNA, 440 nt, Meligethes aeneus
ucccugcaccccacgggagacuacguucuguccacuuccgaagaccaacauugggcguuuuccgacauccguaccggcagacuucugaccaaaguuuccgaucaaucgaacguuccgcugaccaccgcgcagcugcaucccgacggucucaucuucggcaccggaaccggggauucccaggucaaaauuugggauuugaaggagcagagcaacgucgccaacuucacaggacauuccggagcaaucaccacgauuuccuucucggaaaacggguauuacuuggccacugccgccgacgacgcgugcguaaaacuaugggauuugcguaaauuaagaaacuucaaaacucuacaacucgacgaagguuaccaaaucaaagauuuguguuucgaucaaagcggaaccuauuuggccguugccggaacugacgucaggguuua

SEQ ID NO: 15 - RNA, 306 nt, Meligethes aeneus
ggcguuguguugugccauuucgaacgaaguuccggagcauccagugguuucuccuucauccggaguaauauucgagagaagaauaaucgaaaaauacauacaggagaauggaguggacccuauaaguggaaaagaagucgcuauagaugaauuaauagaaaucaaaacgccuccaauuguaaaaccaaagccaccaagugcuacuagcaucccagcuacauuaaaacuacugcaggaugaaugggaugcgguuaugcuguacaguuuuacacaacggcagcaauuacaaacagcgcgacaggaauu

SEQ ID NO: 16 - RNA, 837 nt, Artificial Sequence
  prp19 reg1 hpRNA
ggcguuguguugugccauuucgaacgaaguuccggagcauccagugguuucuccuucauccggaguaauauucgagagaagaauaaucgaaaaauacauacaggagaauggaguggacccuauaaguggaaaagaagucgcuauagaugaauuaauagaaaucaaaacgccuccaauuguaaaaccaaagccaccaagugcuacuagcaucccagcuacauuaaaacuacugcaggaugaaugggaugcgguuaugcuguacaguuuuacacaacggcagcaauuacaaacagcgcgacaggaauugacuaguaccgguugggaaagguauguuucugcuucuaccuuugauauauauauaauaauuaucacuaauuaguaguaauauaguauuucaaguauuuuuuucaaaauaaaagaauguaguauauagcuauugcuuuucuguaguuuauaaguguguauauuuuaauuuauaacuuuucuaauauaugaccaaaacauggugaugugcagguugauccgcgguuaaauuccugucgcgcuguuuguaauugcugccguuguguaaaacuguacagcauaaccgcaucccauucauccugcaguaguuuuaauguagcugggaugcuaguagcacuugguggcuuugguuuuacaauuggaggcguuuugauuucuauuaauucaucuauagcgacuucuuuuccacuuauaggguccacuccauucuccuguauguauuuuucgauuauucuucucucgaauauuacuccggaugaaggagaaaccacuggaugcuccggaacuucguucgaaauggcacaacacaacgcc

SEQ ID NO: 17 - RNA, 21 nt, Meligethes aeneus
ggcguuguguugugccauuuc

SEQ ID NO: 18 - RNA, 22 nt, Meligethes aeneus
gaacgaaguuccggagcaucca

SEQ ID NO: 19 - RNA, 23 nt, Meligethes aeneus
gugguuucuccuucauccggagu

SEQ ID NO: 20 - RNA, 21 nt, Meligethes aeneus
aauauucgagagaagaauaau

SEQ ID NO: 21 - RNA, 22 nt, Meligethes aeneus
cgaaaaauacauacaggagaau

SEQ ID NO: 22 - RNA, 23 nt, Meligethes aeneus
ggaguggacccuauaaguggaaa

SEQ ID NO: 23 - RNA, 21 nt, Meligethes aeneus
agaagucgcuauagaugaauu

SEQ ID NO: 24 - RNA, 22 nt, Meligethes aeneus
aauagaaaucaaaacgccucca

SEQ ID NO: 25 - RNA, 23 nt, Meligethes aeneus
auuguaaaaccaaagccaccaag

SEQ ID NO: 26 - RNA, 21 nt, Meligethes aeneus
ugcuacuagcaucccagcuac

SEQ ID NO: 27 - RNA, 22 nt, Meligethes aeneus
auuaaaacuacugcaggaugaa

SEQ ID NO: 28 - RNA, 23 nt, Meligethes aeneus
ugggaugcgguuaugcuguacag

SEQ ID NO: 29 - RNA, 21 nt, Meligethes aeneus
uuuuacacaacggcagcaauu

SEQ ID NO: 30 - RNA, 22 nt, Meligethes aeneus
uacaaacagcgcgacaggaauu

SEQ ID NO: 31 - RNA, 23 nt, Meligethes aeneus
ggcguuguguugugccauuucga